SEQUENCE LISTING (17 sequences)

SEQ ID NO: 1 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 415 aa, Homo sapiens
YNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 3 - protein, 294 aa, Homo sapiens
AYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSY

SEQ ID NO: 4 - protein, 294 aa, Mus musculus
AYTDETFKTRETIQHESGLLGPLLYGEVGDTLLIIFKNQASRPYNIYPHGITDVSPLHARRLPRGIKHVKDLPIHPGEIFKYKWTVTVEDGPTKSDPRCLTRYYSSFINPERDLASGLIGPLLICYKESVDQRGNQMMSDKRNVILFSIFDENQSWYITENMQRFLPNAAKTQPQDPGFQASNIMHSINGYVFDSLELTVCLHEVAYWHILSVGAQTDFLSIFFSGYTFKHKMVYEDTLTLFPFSGETVFMSMENPGLWVLGCHNSDFRKRGMTALLKVSSCDKSTSDYYEEIY

SEQ ID NO: 5 - protein, 294 aa, Pig
AYTDVTFKTRKAIPYESGILGPLLYGEVGDTLLIIFKNKASRPYNIYPHGITDVSALHPGRLLKGWKHLKDMPILPGETFKYKWTVTVEDGPTKSDPRCLTRYYSSSINLEKDLASGLIGPLLICYKESVDQRGNQMMSDKRNVILFSVFDENQSWYLAENIQRFLPNPDGLQPQDPEFQASNIMHSINGYVFDSLQLSVCLHEVAYWYILSVGAQTDFLSVFFSGYTFKHKMVYEDTLTLFPFSGETVFMSMENPGLWVLGCHNSDLRNRGMTALLKVYSCDRDIGDYYDNTY

SEQ ID NO: 6 - protein, 133 aa, Homo sapiens
SYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 7 - protein, 133 aa, Mus musculus
SYVTPICVANREYTNIFLKFGSGYVSGWGKVFNKGRHASILQYLRVPLVDRATCLRSTTFTTYNNMFCAGYREGGKDSCEGDSGGPHVTEVEGTSFLTGIISWGEECAMKGKYGIYTKVSRYVNWIKEKTKLT

SEQ ID NO: 8 - protein, 133 aa, Dog
SYVTPICIADREYSNIFLKFGSGYVSGWGRVFNKGRSASILQYLKVPLVDRATCLRSTKFTIYNNMFCAGFHEGGKDSCQGDSGGPHVTEVEGISFLTGIISWGEECAMKGKYGIYTKVSRYVNWIKEKTKLT

SEQ ID NO: 9 - protein, 28 aa, Homo sapiens
AYTDETFKTREAIQHESGILGPLLYGEV

SEQ ID NO: 10 - protein, 21 aa, Homo sapiens
DQRGNQIMSDKRNVILFSVFD

SEQ ID NO: 11 - DNA, 19 bp, Homo sapiens
mtakvsscdkntgdyydsy

SEQ ID NO: 12 - protein, 21 aa, Homo sapiens
DSLQLSVCLHEVAYWYILSIG

SEQ ID NO: 13 - protein, 21 aa, Homo sapiens
GKYGIYTKVSRYVNWIKEKTK

SEQ ID NO: 14 - protein, 21 aa, Homo sapiens
CLRSTKFTIYNNMFCAGFHEG

SEQ ID NO: 15 - protein, 16 aa, Homo sapiens
KVSRYVNWIKEKTKLT

SEQ ID NO: 16 - protein, 21 aa, Homo sapiens
SYVTPICIADKEYTNIFLKFG

SEQ ID NO: 17 - protein, 8 aa, Homo sapiens
SVDQRGNQ

(19) United States
(12) Patent Application Publication
(10) Pub. No.: US 2004/0126856 A1
(43) Pub. Date: Jul. 1, 2004

(54) Factor ixa: factor Vllla interaction and methods therefor

(76) Inventors: Paul S. Bajaj, St. Louis, MO (US); Philip J Fay, Rochester, NY (US)

Correspondence Address: THOMPSON COBURN, LLP, ONE US BANK PLAZA, SUITE 3500, ST LOUIS, MO, 63101, US

(21) Appl. No.: 10/466,998

(22) PCT Filed: Jan. 23, 2002
(86) PCT No.: PCT/US02/01724

(51) Int. Cl. C12N 9/99; G01N 33/53; C07K 16/36

(52) U.S. Cl. 435/184; 530/388.25

(57) ABSTRACT

Novel agents that inhibit the interaction of factor VIIIa with factor IXa in newly discovered regions of interaction, Region 2 and Region 3, are disclosed. The novel polypeptides or derivatives of polypeptides prevent activation of factor X and have anti-coagulation activity. The agents include polypeptides or polypeptide derivatives that are homologous to factor VIIIa or factor IXa in Region 2 and/or Region 3, as well as agents that are not homologous, such as antibodies Region 2 or Region 3. Pharmaceutical compositions comprising the agents are also disclosed. Methods of treatment are also disclosed, comprising the step of determining whether the compound displaces the interaction of the above agent from factor VIII or factor IX. Methods for preventing coagulation in a blood sample are also disclosed. These methods comprise adding the above agent to the sample.

### REFERENCE TO GOVERNMENT GRANT

This invention was made with government support under National Institutes of Health Grants HL36365, HL30616 and HL38199. The Government has certain rights in the invention.

### SEQUENCE LISTING

A paper copy of the sequence listing and a computer readable form of the same sequence listing are appended below and herein incorporated by reference. The information recorded in computer readable form is identical to the written sequence listing, according to 37 C.F.R. 1.821 (f).

### BACKGROUND OF THE INVENTION

(1) Field of the Invention

This invention relates generally to the prevention of coagulation. More particularly, this invention relates to compositions and methods for preventing coagulation by inhibiting binding of factor IXa to factor VIIIa, and applications utilizing these compositions and methods, including treating patients in need of anti-coagulants, and preventing coagulation in blood samples.

(2) References Cited

Ahmed et al. (1995) Biochem. J. 310, 427431.

Ardissino et al. (2000) Clin. Chim. Acta 291, 235-240.

Arni et al. (1994) Chem. Phys. Lipids 67/68, 59-66.

Astermark et al. (1994) J. Biol. Chem. 269, 3682-3689.

Bajaj, S. P. (1999) Thrombos. Haemostas. 82, 218-225.

Bajaj, S. P., and Birktoft, J. J. (1993) Methods Enzymol. 222, 96-128.

Bajaj, S. P., and Joist, J. H. (1999) Semin. Thrombos. Hemostas. 25, 407-418.

Bajaj et al. (1981) Prep. Biochem. 11, 397-412.

Bajaj et al. (1992) Proc. Natl. Acad. Sci. USA 89, 152-156.

Banner et al. (1996) Nature 380, 41-46.

Bradford, M. M. (1976) Anal. Biochem. 72, 248-254.

Brandstetter et al. (1995) Proc. Natl. Acad. Sci. U.S.A. 92, 9796-9800.

Breedveld (2000) Lancet 355, 735-740.

Caprino and Han (1972) J Org. Chem. 37, 3404.

Carmeliet and Collen (1996) Semin. Thromb. Hemost. 22, 525-542.

Cheng, Y.-C., and Prusoff, W. H. (1973) Biochem. Pharmacol. 22, 3099-3108.

Church et al. (1984) Proc. Natl. Acad. Sci. USA 81, 6934-6937.

Clowes (1997) Thromb. Haemost. 78, 605-610.

Cortese et al. (1995) Curr. Opin. Biotechnol. 6, 73-80.

Cortese et al. (1995) Curr. Opin. Biotechnol. 7, 616-621.

Craig, D. A., (1993) Trends Pharmacol. Sci. 6, 68-71.

Curtis et al. (1994) J. Biol. Chem. 269, 6246-6251.

Davie et al. (1991) Biochemistry 29, 10363-10370.

DiScipio et al. (1978) J. Clin. Invest. 61, 1528-1538.

DiScipio et al. (1999) Structure 7, 989-996.

Fay, P. J., and Koshibu, K. (1998) J. Biol. Chem. 273, 19049-19054.

Fay, P. J., and Scandella, D. (1999) J. Biol. Chem. 274, 29826-29830.

Fay et al. (1991a) J. Biol. Chem. 266, 8957-8962.

Fay et al. (1991b) J. Biol. Chem. 266, 20139-20145.

Fay et al. (1993) J. Biol. Chem. 268, 17861-17866.

Fay et al. (1994) J. Biol. Chem. 269, 20522-20527.

Feuerstein et al. (1999) Arterioscler. Thromb. Vasc. Biol. 19, 2554-2562.

Freedman et al. (1996) J. Biol. Chem. 272, 22037-22045.

Freidinger (1999) Curr. Opin. Chem. Biol 3:395-406.

Galfre and Milstein (1981) Methods Enzymol. 73 (Pt. B), 3-46.

Gallo et al. (1998) Circulation 97, 581-588.

Green et al. (2000). Haemophilia B: database of point mutations and short additions and deletions. http://www.umds.ac.uk/molzen/intro.htm.

Guex et al. (1999) Trends Biochem. Sci. 24, 364-367.

Halfman, C. J. (1981) Methods Enzymol. 74, 481-408.

Hamaguchi et al. (1994) Blood 84, 1837-1842.

Hemostasis Research Group at the MRC Clinical Sciences Center, Imperial College Medical School (2000). Hemophilia. A mutation database. http://europium.csc.mrc.ac.uk/usr/WWW/WebPages/main.dir/main.htm.

Hopfner et al. (1999) Structure 7, 989-996.

Husten et al. (1987) J. Biol. Chem. 262, 12953-12966.

Igarashi et al. (1993) Mol Cell Biol 13, 1634-1640.

Kieber-Emmons et al. (1997) Curr. Opin. Biotechnol. 8:435-441.

Kolkman et al. (1999) Biochem. J. 339, 217-221.

Koszelak et al. (2000) J. Biol. Chem. In Press.

Lapan, K. A., and Fay, P. J. (1997) J. Biol. Chem. 272, 2082-2088.

Laskowski et al. (1993) J. Appl. Cryst. 26, 283-291.

Lenting et al. (1996) J. Biol. Chem. 271,25332-25337.

Lollar, P., and Parker, C. G. (1989) Biochemistry 28, 666-674.

Lollar, P., and Parker, G. G. (1990) J. Biol. Chem. 265, 1688-1692.

Lollar et al. (1994) J. Clin. Invest. 93, 2497-2504.

Lottenberg et al. (1981) Methods Enzymol. 80, 341-361.

Mann, K. G. (1999) Thrombos. Haemostas. 82, 165-174.

Mann et al. (1988) Ann. Rev. Biochem. 57, 915-956.

Mathur, A., and Bajaj, S. P. (1999) J. Biol. Chem. 274, 18477-18486.

Mathur et al. (1997) J. Biol. Chem. 272, 23418-23426.

Mertens, K. (1996) J. Biol. Chem. 271, 1935-1940.

Mertens et al. (1999) Thrombos. Haemostas. 82, 209-217.

Merrifeld (1963) J. Am. Chem. Soc. 85, 2149.

Mimms et al. (1981) Biochemistry 20, 833-840.

Navia and Peattie (1993) Trends Pharm. Sci. 14:189-195

Nicholls et al. (1991) Proteins 11, 281-296.

O'Brien et al. (1995) J. Biol. Chem. 270, 27087-27092.

Ohno et al. (1980) Thrombosis Research 19, 579-588.

Oltrona et al. (1997) Circulation 96, 646-652.

Pemberton et al. (1997) Blood 89, 2413-2421.

Pratt et al. (1999) Nature (London) 402,439-442.

Qabar et al. (1996) Farmaco 51, 87-96.

Rao et al. (1995) Cell 82, 131-141.

Peitsch (1996) Biochem. Soc. Trans. 24, 274-279.

Regan et al. (1994) J. Biol. Chem. 269, 9445-9452.

Ripka et al. (1998) Curr. Opin. Chem. Biol. 2:441-452.

Sabharwal et al. (1997) J. Biol. Chem. 272, 22037-22045.

Sarkar et al. (1990) Genomics 6, 133-134.

Schwartz and Moawad (1997) Ann. Vasc. Surg. 11, 189-199.

Stoylova et al. (1999) J. Biol. Chem. 274, 36573-36578.

Toole et al. (1984) Nature 312, 342-347.

Toschi et al. (1997) Circulation 95, 594-599.

van Dieijen et al. (1981) J. Biol. Chem. 256, 3433-3442.

Vehar et al. (1984) Nature 312, 337-342.

Winter and Milstein (1991) Nature 349, 293-299.

Wood et al. (1984) Nature 312, 330-337.

Yla-Herttuala and Martin (2000) Lancet 355, 213-222.

Yoshitake et al. (1985) Biochemistry 24, 3736-3750.

Zaitseva et al. (1996) J. Biol. Inorg. Chem. 1, 15-23.

Zhong et al. (1994) Proc. Natl. Acad. Sci. U.S.A. 91, 3574-3578.

Zhong, D., and Bajaj, S. P. (1993) Biotechniques 15, 874-878.

(3) Description of the Related Art

Two common causes of abnormal bleeding are deficiencies of factor VII (hemophilia A) or factor IX (hemophilia B). Factor IX, a vitamin K-dependent protein, is synthesized by hepatocytes as a precursor molecule of 461 residues containing a 28 residue signal propeptide and an 18 residue leader propeptide (Yoshitake et al., 1985). During biosynthesis, the nascent protein undergoes several posttranslational modifications, resulting in a single-chain protein consisting of 415 amino acids and containing 17% carbohydrate by weight (DiScipio et al., 1978). The mature protein circulates in blood as a zymogen of Mr 57,000.

Factor IX is activated during physiologic clotting to the two-chain, disulfide-linked serine protease, factor IXa, by VIIa/Ca²⁺/tissue factor (TF) or by factor XIa/Ca²⁺ (Davie et al., 1991). The domain organization of factor IXa is similar to those of the other two enzymes (factors VIIa and Xa) involved in the TF-induced coagulation and to that of an anticoagulant enzyme termed activated protein C. The light chain of IXa consists of an amino-terminal γ-carboxyglutamic acid domain (“Gla domain”, residues 1-40 out of which 12 are γ-carboxyglutamic acid residues), a short hydrophobic segment (residues 41-46), and two epidermal growth factor (EGF)-like domains (EGF1 residues 47-85, and EGF2 residues 86-127) whereas the heavy chain contains the carboxy-terminal serine protease domain with trypsin-like specificity (Davie et al., 1991; Brandstetter et al., 1995). Activation peptide (AP) of residues 145-180, which is released upon conversion of factor IX to IXa, is rich in carbohydrate and is the least conserved region in IX from different species (Sarkar et al., 1990). Factor IXa hence formed converts factor X to Xa in the coagulation cascade; for a biologically significant rate, this reaction requires Ca²⁺, phospholipid and factor VIIIa. The amino acid sequences of factor VIIIa and factor IXa are provided herein in the sequence listing as SEQ ID NO:1 and SEQ ID NO:2, respectively.

Based upon the crystal structure of the Gla domain of factor VIIa (Banner et al., 1996) and the Ca²⁺-binding properties of factor X (Sabharwal et al., 1997), it would appear that this domain in IXa possesses several low to intermediate affinity Ca²⁺-binding sites. In addition, the EGF1 and the protease domain each possess one high affinity Ca²⁺-binding site (Rao et al., 1995; Bajaj et al., 1992). The Ca²⁺-loaded conformer of the Gla domain binds to phospholipid vesicles (Freedman et al., 1996) and the EGF1 domain of IX is required for its activation by VIIa/Ca²⁺/TF (Zhong et al., 1994). Further, Ca²⁺-binding to the EGF1 domain has been reported to promote enzyme activity and factor VIIIa binding (Lenting et al., 1996). For proper binding of IXa to PL and VIIIa, all of the Ca²⁺-sites in IXa must be filled (Bajaj, 1999; Mertens et al., 1999). The role of the EGF2 domain is not clear but may be involved in binding to platelets and in factor X activation (Ahmed et al., 1995). Finally, the protease domain is thought to play a primary role in binding to factor VIIIa (Astermark et al., 1994; O'Brien et al., 1995; Bajaj et al., 1993).

It has been demonstrated that mutations in the protease domain Ca²⁺-binding ligands decrease the affinity of factor IXa for factor VIIIa by ˜15-fold and that proteolysis at R318-S319 [residues 150-151 in the chymotrypsin numbering system] in the autolysis loop results in a further decrease in this interaction by ˜8-fold (Mathur et al., 1997, J. Biol. Chem. 272, 23418-23426). Since residues in the protease domain Ca²⁺-binding loop as well as those in the autolysis loop may not directly participate in binding to factor VIIIa (Hamaguchi et al., 1994), Ca²⁺ binding to the protease domain and integrity of the autolysis loop stabilize yet another region in this domain of factor IXa that directly interacts with factor VIIIa. This region has recently been identified as the 330 helix of factor IXa, comprising residues L330-R-338, corresponding to residues 162-170 using the chymotrypsin numbering system (Mathur and Bajaj, 1999; Bajaj, 1999).

Factor VIII is synthesized as a single chain molecule containing several domains (A1-A2-B-A3-C1-C2) (Vehar et al., 1984), with a molecular mass of approximately 300 kDa (Wood et al., 1984; Toole et al., 1984). The A domains are homologous to the ceruloplasmin domains and to the A domains of factor Va (Pemberton et al., 1997), whereas the C domains are homologous to the galactose lipid binding domain and to the regions within neuraminidase (Pratt et al., 1999). Factor VIII circulates as a divalent metal ion-dependent, noncovalent heterodimer resulting from proteolytic cleavage at the B/A3 junction that generates a heavy chain (A1-A2-B) and a light chain (A3-C1-C2). This procofactor form is cleaved by thrombin at R372-S373, R740-S741, and R1689-S1690 to yield factor VIIIa, a heterotrimer composed of A1, A2 and A3-C1-C2 subunits (Lollar and Parker, 1989; Fay et al., 1991a). The A1 and A3-C1-C2 subunits remain associated with a divalent metal ion dependent linkage whereas A2 subunit is weakly associated with the A1 and A3-C1-C2 dimer (Lollar and Parker, 1990; Fay et al., 1991b). While intact factor VIIIa is required for maximal enhancement of factor IXa activity, recent results have shown that the isolated A2 subunit stimulates factor IXa by ˜100-fold (Fay and Koshibu, 1998). However, peptides of A2 residues S558-Q565, K556-N564, and Q561-D569 inhibit factor Xa generation in purified systems (Fay et al., 1994; Fay and Koshibu, 1998).

Ca²⁺-dependent assembly of factor IXa and factor VIIIa on a suitable PL surface is essential for hemostasis since defects or deficiency in the proteins result in severe bleeding diatheses, namely, hemophilia A (factor VIII deficiency) or hemophilia B (factor IX deficiency) (Hemostasis Research Group, 2000; Green et al., 2000). In this assembly, Ca²⁺-loaded form of the Gla domain of IXa binds to PL (Freedman et al., 1996) whereas EGF1³/EGF2 region(s) and the protease domain are thought to interact with A3 and A2 domains of VIIIa, respectively (Fay and Koshibu, 1998; Lenting et al., 1996). VIIIa in this assembly is thought to be anchored to the PL surface via C2 domain (Pratt et al., 1999). Binding of substrate factor X to this IXa/VIIIa assembly may be partly mediated through the A1 domain of VIIIa (Lapan and Fay, 1997). Thus, although it has been shown that helix 330 of IXa and A2 domain of VIIIa interact with each other, little is known regarding the interface region(s) between these two modules, or other areas of interaction between factor VIIIa and factor IXa.

The identification of other sites of interaction between factor VIIIa and factor IXa would be useful for devising methods and reagents for inhibiting clotting in vitro and in vivo.

### SUMMARY OF THE INVENTION

In accordance with the present invention, the inventors have succeeded in identifying two new areas in factor VIIIa and factor IXa that interact during factor X activation. The amino acid designations used herein are based upon a human factor VIII sequence as depicted in SEQ ID NO:1 and a human factor IX sequence as depicted in SEQ ID NO:2. These two new areas are identified herein as Region 2 and Region 3. Region 2 comprises the interaction between N346 (178 by the chymotrypsin numbering system) of factor IXa and E455 and K570 of factor VIIIa, and the interaction between R403 (233 chymotrypsin) of factor IXa and E633 of factor VIIIa. Region 3 comprises the interaction between K293 (126 chymotrypsin) of factor IXa and D712 of factor VIIIa, and the interaction between E410 (240 chymotrypsin) of factor IXa and K713 of factor VIIIa. By utilizing this knowledge, novel compositions and methods for inhibiting coagulation are disclosed.

Thus, in some embodiments, the present invention is directed to an agent that specifically inhibits the interaction of factor VIIIa with factor IXa in Region 2 and/or Region 3 without activating factor X. Preferably, the agent inhibits coagulation. The agent can be a polypeptide or a derivative thereof, where the polypeptide comprises an amino acid sequence of at least 3 contiguous amino acids homologous to (a) a sequence in factor VIII comprising E445, D570, E633, D712, or K713; or (b) a sequence in factor IX comprising N346 (chymotrypsin 178), R403 (chymotrypsin 233), K293 (chymotrypsin 126), or E410 (chymotrypsin 240). Preferably, the amino acid sequence is at least 5 amino acids long, more preferably, 10 amino acids long. The agent also can comprise at least two amino acids identified in part (a) or (b) above. For example, the amino acid sequence can comprise a sequence selected from the group consisting of factor VIII sequences E445 through K570; E445 through E633, E445 through K713, K570 through E633, K570 through K713, and E633 through K713 of factor VIIIa, and factor IX sequences K293 through N346; N346 through R403; R403 through E410; K293 through R403; N346 through E410; and K293 through E410. The agent can also be a nonpeptidomimetic of these amino acid sequences.

The agent can also be a peptide comprising a sequence from any one of (a) region 2 or 3 of factor VIII (SEQ ID NO:3), (b) region 2 or 3 of factor IX (SEQ ID NO:6), (c) a sequence that is at least 88% identical to SEQ ID NO:3, and (d) a sequence that is at least 88% identical to SEQ ID NO:6, wherein the sequence is at least three amino acids long. The most preferred peptides comprise an amino acid sequence of any one of SEQ ID NOS:9-16.

The agent can also be a non-homologous binding polypeptide. Preferably, the non-homologous binding polyeptide agent has an antibody binding site that specifically binds to factor VIIIa or factor IXa in Region 2 or Region 3. The antibody binding site preferably specifically binds to the amino acid sequence of the agents above that are homologous to factor VIIIa or factor IXa in Region 2 or Region 3. In preferred embodiments, the agent is an antibody, most preferably a monoclonal antibody, particularly a humanized monoclonal antibody.

In additional embodiments, the present invention is directed to a polynucleotide encoding an amino acid sequence homologous to any one of the above-described agents, where the polynucleotide is operably linked to a control sequence that allows the polynucleotide to be translated in a mammalian cell.

In other embodiments, the present invention is directed to a composition that induces coagulation. The composition comprises the portions of the amino acid sequence of factor VIIIa that interact with factor IXa, or derivatives thereof. The composition could comprise the entire portion of the factor VIIIa amino acid sequence that encompasses the factor IXa-interacting portions (E440-K713 of SEQ ID NO:1), or it could comprise the amino acid fragments that interact with factor IXa connected by linkers designed to align the interacting portions to the proper areas of factor IXa.

The present invention is also directed to pharmaceutical compositions comprising the agents or polynucleotides encoding the agents disclosed above, in a pharmaceutically acceptable excipient. In preferred embodiments, the excipient is suitable for intravenous administration.

Additionally, the present invention is directed to a method of treatment to prevent coagulation in a patient in need thereof. The method comprises administering to the patient any of the polypeptide or polynucleotide agents disclosed above, preferably as the above-described pharmaceutical compositions. The method is particularly useful for patients suffering from a cardiovascular disorder, where the preferred disorders are thrombosis, atherosclerosis and restenosis. In most preferred embodiments, the pharmaceutical composition is administered intravenously.

The present invention is also directed to a method for identifying a compound having anti-coagulation activity. The method comprises determining whether a compound displaces the interaction of any of the above-described agents to factor VIIIa or factor IXa. Preferably, the agent is labeled with a detectable marker, most preferably a fluorescent marker, a radioactive marker, and a spin label.

In additional embodiments, the present invention is directed to a method of preventing coagulation in a blood sample. The method includes adding any of the above-described agents to the sample.

### BRIEF DESCRIPTION OF THE DRAWINGS

FIG. 1 is a graph depicting the effect of the isolated A2 subunit of factor VIIIa on the rate of activation of factor X by various factor IXa proteins. The rate of formation of factor Xa by each factor IXa protein was measured as described in the Example. The reaction mixtures contained 5 nM factor IXa, 250 nM factor X and various concentrations of A2 subunit. The buffer used was TBS/BSA, pH 7.5 containing 25 μM PL and 5 mM CaCl₂. The proteins used are: IXa_{WT} (●), IXa_{PCEGF1} (◯), IXa_{R333Q} (▴), and IXa_{VIIhelix} (Δ). The data were fitted to a single site binding equation (Eq 1).

FIG. 2 is a graph depicting the effect of factor X concentration on the EC₅₀ (functional Kd) of the interaction of the A2 subunit with IXa_{WT} or IXa_{PCEGF1}. The EC₅₀ of the interaction of factor IXa_{WT} (●) or factor IXa_{PCEGF1} (◯) with the A2 subunit was determined at various concentrations of factor X. Each point (EC₅₀) shown is the concentration of free A2 subunit (y-axis) providing 50% of the Vmax. Each EC₅₀ value was obtained from a direct plot (similar to FIG. 1) of factor Xa generation at various concentrations of the A2 subunit and a constant concentration of factor X. The factor IXa concentration in each experiment was fixed at 5 nM. The buffer used was TBS/BSA, pH 7.5 containing 25 μM PL and 5 mM CaCl₂. Factor Xa concentration was measured by S-2222 hydrolysis.

FIG. 3 is a graph depicting the abilities of various dEGR-IXa proteins to inhibit factor IXa:A2 subunit interaction as measured by a decrease in factor Xa generation in the tenase system. The reaction mixtures contained 100 nM IXa_{WT}, 30 nM A2 subunit, 250 nM factor X, 25 μM PL, and various concentrations of dEGR-IXa proteins in TBS/BSA, pH 7.5 containing 5 mM CaCl₂. Factor Xa generation was measured by S-2222 hydrolysis. The value of slope factor, s, was 0.9±0.1 indicating a single affinity binding site between the interacting proteins. The curves represent best fit of the data to the IC₅₀ four-parameter logistic equation (Eq 2). The proteins used are: dEGR-IXa_{WT} (●), dEGR-IXa_{PCEGF1} (◯), dEGR-Xa_{R333Q} (▴) and dEGR-IXa_{VIIhelix} (Δ).

FIG. 4 is a graph depicting the effect of the A2 subunit on the fluorescence emission intensity of dEGR-IXa proteins. Reactions (160 μL) were titrated with A2 subunit in buffer containing 20 mM Hepes, pH 7.2, 100 mM NaCl, 5 mM CaCl₂, 0.01% Tween, 200 μg/ml BSA and 100 μM PL vesicles. Fluorescence emission intensity of each dEGR-IXa (220 nM) at a given A2 subunit concentration was determined as described in the Example. Data are presented as F/F₀, where F₀ is emission intensity in the absence of A2 and F is the intensity at a given A2 subunit concentration. Symbols are dEGR-IX_{WT} (▴), dEGR-IXa_{PCEGF1} (●), dEGR-IXa_{R333Q} (♦), and dEGR-IX_{VIIhelix} (▪).

FIG. 5 is a graph depicting the ability of the 558-565 A2 peptide to inhibit the interaction of various factor IXa proteins with the A2 subunit. The reaction mixture for factor IXa_{WT} (●) or factor IXa_{PCEGF1} (◯) contained 100 nM of factor IXa protein, 30 nM A2 subunit, 250 nM factor X, 5 mM CaCl₂, and 25 μM PL in TBS/BSA, pH 7.5. The reaction mixture for factor IXa_{R333Q} ( ) contained 300 nM of factor IXa instead of 100 nM used for factor IXa_{WT} or factor IXa_{PCEGF1}; the concentrations of other components were unchanged. Factor Xa generation was determined by S-2222 hydrolysis, and the curves represent best fit to the IC₅₀ four-parameter logistic equation (Eq. 2). The value of slope factor, s, was 0.9±0.1 indicating a single affinity binding site between the various IXa proteins and the A2 peptide.

FIG. 6 depicts an interface model between the factor IXa protease domain and the A2 subunit of factor VIIIa. The coordinates for the human factor IXa structure are from the Brookhaven Protein Data Bank (PDB code 1RFN) and the coordinates for the A1, A2, and A3 subunits of factor VIIIa (Pemberton et al., 1997) are based upon homology models built using ceruloplasmin coordinates (PDB code 1KCW). A, Schematic representation of the interface model. The ribbon structure for each protein is depicted. The IXa protease domain is shown in light blue and the EGF2 domain is shown in red. The A1 subunit is in yellow, the A2 subunit is in magenta with residues 484-509 in white, and the A3 subunit is in cyan with the C-terminal in red. The Gla and the EGF1 domains of factor IXa and the C1 and C2 domains of factor VIIIa are not shown. The interface residues of the factor IXa protease domain and of the A2 subunit are shown as CPK space filling models. The molecules are oriented such that the Gla domain of factor IXa and the C2 domain of factor VIIIa are projecting away from the viewer. The Gla domain in factor IXa and the C2 domain of factor VIIIa bind to the PL surface. B, Detailed interface between factor IXa protease domain and the modeled A2 subunit. Only the charged residues that participate in the binding interactions are depicted. The hydrophobic residues that participate in this interaction are discussed in the text. The orientation of the molecules is the same as in A. Chymotrypsin numbering system for the factor IXa protease domain is used. Corresponding factor IX numbering system are 338 (c170), 332 (c164), 333 (c165), 346 (c 178), 403 (c233), 293 (c126) and 410 (c240). Factor IXa residues are labeled light blue and A2 subunit residues are labeled magenta. C, Electrostatic potential between the factor IXa protease domain and the A2 subunit interface as determined using the program GRASP (Nicholls et al., 1991). Blue represents positive, red represents negative and white represents neutral residues.

FIG. 7 depicts an alignment between the sequences depicting regions 2 and 3 of various mammalian factors VIII (A) and IX (B).

### DETAILED DESCRIPTION OF THE INVENTION

The amino acid numbering system that is herein used is based upon the human factor VIII sequence of SEQ ID NO:1 and the human factor IX sequence of SEQ ID NO:2, unless indicated otherwise.

The following abbreviations are used herein: TF, tissue factor; Gla, gamma-carboxyglutamic acid; EGF, epidermal growth factor; PL, phospholipid; BSA, bovine serum albumin; WT, wild type; TBS, Tris-buffered saline; dEGR-ck, dansyl-Glu-Gly-Arg-chloromethyl ketone; dEGR-IXa, IXa inactivated with dEGR-ck; S-2222, benzoyl-Ile-Glu-Gly-Arg-p-nitroanilide; NP, normal plasma; Kd_{A2}, dissociation constant for dEGR-IXa and the A2 subunit; Kd_{peptide}, dissociation constant for factor IXa and the A2 558-565 peptide. The numbers in parentheses with a prefix c (e.g., c57) refers to the chymotrypsin equivalents for the protease domain of factor IXa (Bajaj and Burktoft, 1993).

In accordance with the present invention, it has been discovered that factor VIIIa and factor IXa interact at two regions, Region 2 and Region 3, which have not been previously identified as areas of interaction. Region 2 is defined herein as the interaction between N346 (178 by the chymotrypsin numbering system) of factor IXa and E445 and K570 of factor VIIIa, and the interaction between R403 (c233) of factor IXa and E633 of factor VIIIa. Region 3 is defined herein as the interaction between K293 (c126) of factor IXa and D712 of factor VIIIA, and the interaction between E410 (c240) of factor IXa and K713 of factor VIIIA. These interactions are necessary for normal conversion of factor X to factor Xa by the factor IXa protease domain, as normally occurs in clotting. The skilled artisan would thus expect anything that disrupts these interactions to inhibit clotting. It is also noted that mutations at amino acid residue 403 (c233) of factor IX has been found to cause hemophilia (Green et al. 2000). Similar findings have been shown for Region 1, defined herein as the interaction between Helix 330 (amino acid residues 330-338 [c162-c170]) of factor IXa and residues 558-565 of factor VIIIa. In that region, mutants of factor IXa inhibited the formation of factor Xa in vitro (Example; Mather and Bajaj, 1999), and short peptides with sequences identical to that region of factor VIIIa also inhibited formation of factor Xa (Fay et al., 1994) in vitro.

As used herein, “clotting” or “blood clotting” or “coagulation” means the sequential process by which the multiple coagulation factors of the blood interact in the coagulation cascade, ultimately resulting in the formation of an insoluble fibrin clot.

As used herein, “inhibiting clotting” encompasses effects where clotting is eliminated, as well as where clotting is just reduced to a significant degree. Depending on the desired goal, preferred methods and agents of the present invention will inhibit thrombosis under optimum conditions by at least 10%; more preferably, the inhibition will be at least 25%; even more preferably, at least 50%; even more preferably, at least 75%. The most preferred methods or reagents of the present invention inhibit thrombosis by 90-100% under optimized conditions; however, if desired, the conditions could be adjusted to be suboptimal if lower degrees of inhibition of thrombosis are desired.

Reduction or inhibition of clotting can be measured by any means known in the art. Nonlimiting examples of useful methods to measure clotting include (a) methods that directly measure the interaction of factor VIIIa with factor IXa, for example by measuring changes in dansyl emission intensity (see, e.g., Example 1); (b) methods that measure the result of the factor VIIIa-factor IXa interaction, i.e., formation of factor Xa (TENase activity) (see, e.g., Example 1); and (c) methods that measure rate of thrombosis, such as the well-known thrombin time, prothrombin time, or activated partial thromboplastin time assays. See, generally, Lottenberg et al., 1981, and Ohno et al., 1980.

Thus, in some embodiments, the present invention provides an agent that specifically inhibits clotting by preventing the interaction of a factor VIIIa with a factor IXa in Region 2 or Region 3. In one group of these embodiments, the agent is a polypeptide, or a derivative thereof, that (a) is capable of interacting with factor VIIIa in Region 2 or Region 3 by virtue of its sequence homology with factor IXa, and/or (b) is capable of interacting with factor IXa in Region 2 or Region 3 by virtue of its sequence homology with factor VIIIa. Since these polypeptides or derivatives are homologous to factor VIIIa or factor IXa at regions of interaction, they would be expected to interact with factor IXa or factor VIIIa, respectively, blocking the interaction between the two factors. This would prevent formation of factor Xa and inhibiting clotting.

This group of clotting inhibitors would exclude polypeptides or derivatives that are capable of inducing coagulation to a limited degree by substituting for factor VIIIa or IXa. For example, the A2 region of factor VIII would not be an agent that inhibits clotting by preventing the interaction of factor VIIIa with factor IXa, because the A2 region is capable of inducing coagulation in a TENase reaction mixture by substituting for factor VIIIa (Fay and Koshibu, 1998; see also Example 1). The skilled artisan could easily test any polypeptide or derivative for its ability to induce coagulation by determining whether the polypeptide or derivative is capable of inducing coagulation in a TENase reaction mixture without factor VIIIa or factor IXa.

These polypeptides can be produced by any of several well-known methods, including expressing a clone of a gene that encodes the polypeptide, and chemical synthesis, for example by the classical Merrifeld method of solid phase peptide synthesis (Merrifeld, 1963) or the FMOC strategy on a Rapid Automated Multiple Peptide Synthesis system (DuPont Company, Wilmington, Del.) (Caprino and Han, 1972).

Since Region 2 and Region 3 comprise the amino acids E445, K570, E633, D712 and K713 in factor VIIIa (SEQ ID NO:1) and N346 (178 chymotrypsin), R403 (233 chymotrypsin), K293 (126 chymotrypsin) and E410 (240 chymotyypsin) in factor IXa (SEQ ID NO:2) the inhibitory peptide or derivative preferably comprises an amino acid sequence, or derivative, homologous with factor VIIIa or factor IXa that comprises at least one of those amino acid residues. As such, examples of polypeptides or derivatives that would be useful for the present invention include any amino acid sequence of at least 3 contiguous amino acids homologous to a sequence in factor VIIIa that comprises E445, K570, E633, D712 or K713, or any amino acid sequence of at least 3 contiguous amino acids homologous to a sequence in factor IXa that comprises N346, R403, K293, or E410. In preferred embodiments, the polypeptide or derivative comprises at least 5 amino acids homologous to factor VIIIa or factor IXa; more preferably, the polypeptide or derivative comprises at least 10 amino acids homologous with factor VIIIa or factor IXa.

Preferably, the peptide or derivative is homologous to an amino acid sequence from factor VIIIa or factor IXa that also encompasses other amino acid residues that are involved in the interaction of factor VIIIa with factor IXa, for example other residues from Region 2 or Region 3, residues from Region 1 (encompassing the interaction of the helix 330 [chymotrypsin 162] of factor IXa with residues 558-565 of factor VIIIa—see Example), or residues involved in calcium binding. Examples of amino acid sequences or derivatives that are particularly useful for the present invention include sequences that are derived from region 2 and 3 of factor VIII, which includes human (SEQ ID NO:3), mouse (SEQ ID NO:4), and pig sequences (SEQ ID NO:5), and sequences that are at least 88% identical to SEQ ID NOS:3-5, and sequences that are derived from region 2 and 3 of factor IX, which includes human (SEQ ID NO:6), mouse (SEQ ID NO:7), and dog sequences (SEQ ID NO:8), and sequences that are at least 88% identical to SEQ ID NOS:6-8. It is further envisioned that those sequences comprise residues E445 through K570; residues E445 through E633, residues E445 through K713, residues K570 through E633, residues K570 through K713, and residues E633 through K713 of factor VIIIa, and factor IXa sequences K293 through N346; N346 through R403; R403 through E410; K293 through R403; N346 through E410; and K293 through E410.

A preferred peptide comprises an amino acid sequence that is selected from the list consisting of SEQ ID NO:9, SEQ ID NO:10, SEQ ID NO:11, SEQ ID NO:12, SEQ ID NO:13, SEQ ID NO:14, SEQ ID NO:15 and SEQ ID NO:16.

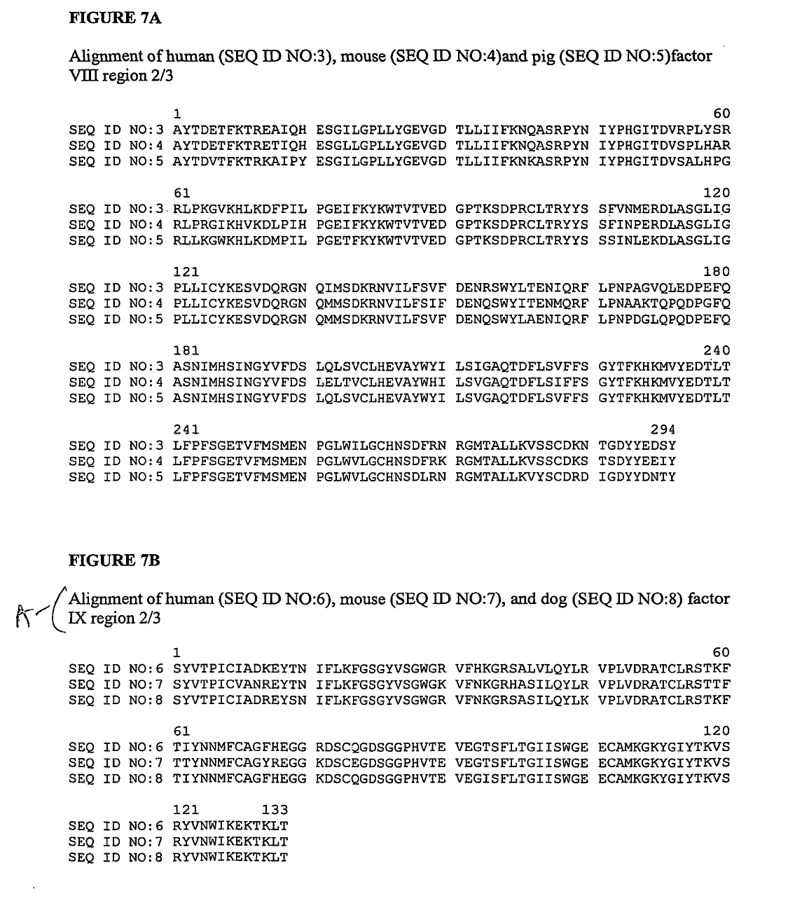

The step wise comparison and alignment of regions 2 and 3 of factors VIII and IX from various mammalian species, according to the ClustalW program or similar sequence alignment program (the parameters of which are detailed below), is depicted in FIG. 7. The proportion of identical amino acids between human and mouse and human and pig or dog was determined from aligned sequences and reported herein as percent identity in Table 1.

TABLE 1

| | | Percent |
|---|---|---|
| | Sequence | amino |
| Species compared | identifiers | acid identity |
| Human v. mouse Factor VIII region 2/3 | SEQ ID NO: 3 v. | 88% |
| | SEQ ID NO: 4 | |
| Human v. pig Factor VIII region 2/3 | SEQ ID NO: 3 v. | 88% |
| | SEQ ID NO: 5 | |
| Human v. mouse Factor IX region 2/3 | SEQ ID NO: 6 v. | 90% |
| | SEQ ID NO: 7 | |
| Human v. dog Factor IX region 2/3 | SEQ ID NO: 6 v. | 95% |
| | SEQ ID NO: 8 | |

Sequence identity or percent identity is intended to mean the percentage of same residues between two. sequences aligned using the Clustal method (Higgins et al, Cabios 8:189-191, 1992) of multiple sequence alignment in the Lasergene biocomputing software (DNASTAR, INC, Madison, Wis.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=10; gap length penalty for multiple alignment=10; k-tuple value in pairwise alignment=1; gap penalty in pairwise alignment=3; window value in pairwise alignment=5; diagonals saved in pairwise alignment=5. The residue weight table used for the alignment program is PAM250 (Dayhoff et al., in Atlas of Protein Sequence and Structure, Dayhoff, Ed., NBRF, Washington, Vol. 5, suppl. 3, p. 345, 1978).

To determine percent sequence identity between two sequences, the number of identical amino acids in the aligned sequences is divided by the total number of amino acids that are compared. The sequence identity between human factor VIII region 2/3 and mouse or pig factor VIII region 2/3 is about 88%.

Based upon the comparison of human region 2/3 sequence to other mammalian homologues of region 2/3, the inventor envisions that the polypeptide or derivative that is capable of inhibiting coagulation comprises a portion of a sequence that is at least 88% identical to SEQ ID NO:3 or a portion of a sequence that is at least 88% identical to SEQ ID NO:6.

The polypeptide or derivative can be of any length, provided it is capable of inhibiting coagulation, and may comprise a sequence homologous to a large portion of factor VIII or factor IX, or may comprise a sequence that is homologous to factor VIII or factor IX at Region 2 or Region 3 fused to a sequence that is not homologous. For example, the polypeptide or derivative can be 5, 10, 15, 20, 25, 30, 35, 40, 50, 60, 70, 80, 90, 100, 120, 150, 200, or more amino acids or amino acid derivatives long, or any length between those values.

As used herein, the term “derivative” includes any non-peptide compound, including peptidomimetics or nonpeptidomimetics, that can substitute for a particular amino acid or polypeptide. Based on the structural features of the critical amino acid sequence of the peptides of the present invention that permit the interaction of the peptide to factor VIIIa or factor IXa, one can develop these non-peptide derivatives that are capable of binding to factor VIIIa or factor IXa and that inhibit coagulation. Thus, a non-peptide derivative includes any non-peptide chemical compound that can interact with factor VIIIa or factor IXa at Region 2 or Region 3 to inhibit clotting.

The techniques for development of peptidomimetics and nonpeptidomimetics are well known in the art. See for example, Navia and Peattie, 1993; Ripka et al., 1998; Kieber-Emmons et al., 1997; Freidinger, 1999; Qabar et al., 1996. Typically this involves identification and characterization of the protein target as well as the protein ligand using X-ray crystallography and nuclear magnetic resonance technology. In the case of the factor VIIIa binding domain on factor IXa, both factors have been sequenced and cloned (Wood et al., 1984; Vehar et al., 1984; Yoshitake et al., 1985). Additionally, the X-ray structure of factor IXa has been determined (Brandstetter et al., 1995) and modeling studies have elucidated characteristics of the factor VIIIa and factor IXa binding site (disclosed herein and in Mather and Bajaj, 1999). Using information learned from the structure of factor VIIIa and its polypeptide ligand, a pharmacophore hypothesis is developed arid compounds are made and tested in a routine assay system. The test compound can then be evaluated by, for example, binding to factor VIIIa or IXa, e.g., by electrophoretic mobility shift assays (Igarashi et al., 1993) or an assay system utilizing co-precipitation of the ligand and factor VIIIa or IXa. Alternatively, the compound can also be tested functionally by methods known in the art, e.g., by its ability to reduce or abolish activity of factor VIIIa or factor IXa in a coagulation based assay or in factor X activation assay. See, e.g., Examples 1 and 2 for such methods. As is well known, peptidomimetics and nonpeptidomimetics are often superior to analogous peptides in therapeutic applications because the mimetics are generally more resistant to digestion than peptides.

Additionally, included within the derivatives contemplated as part of the invention are the polypeptides disclosed above, wherein individual amino acids in the claimed sequence are substituted with linkers which are not amino acids but which allow other amino acids in the sequence to be spaced properly to allow binding to factor VIIIa. For example, the C of the sequence MTALLKVSSCDKNTGDYYEDSY (SEQ ID NO:11) can be replaced with a linker to allow the other areas of the sequence to align properly with Region 3 of factor IXa. Use of such linkers is well known in the art and their design in this context would not require undue experimentation.

Another group of agents that can inhibit clotting according to the invention is the group of agents that comprise non-homologous binding polypeptides. These polypeptides are not homologous to factor VIII or factor IX at Region 2 or Region 3, but are able to bind to factor VIII or factor IX at those regions. This group of agents consists of (a) polypeptides that bind to Region 2 or Region 3 through an antibody binding site and (b) polypeptides that do not bind to Region 2 or Region 3 at an antibody binding site. The latter polypeptides can be identified, e.g., by random peptide libraries, such as phage display libraries (Cortese et al., 1995; Cortese et al., 1996). Peptidomimetics or nonpeptidomimetics that are derivatives of these peptides, prepared by methods known in the art, are also envisioned as being within the scope of the present invention.

In preferred embodiments, the non-homologous binding peptide comprises an antibody binding site that specifically binds to factor VIIIa or factor IXa in Region 2 or Region 3. Due to their ease of preparation, these agents are preferably antibodies or antibody fragments such as FAb or F(Ab)₂ fragments, but other types of polypeptides comprising antibody binding sites can be prepared by known methods (see, e.g., Winter and Milstein, 1991).

These agents comprising an antibody binding site that specifically binds to factor VIII or IX at Region 2 or Region 3 would be expected to inhibit TENase activity and coagulation, since antibodies to other regions of factor IX have been shown to have such an effect in vitro and in vivo (Feuerstein, 1999).

The antibodies of these embodiments can be polyclonal or, preferably, monoclonal antibodies, which can be prepared by, e.g., the well-known hybridoma method (Galfre and Milstein, 1981) or by recombinant methods (Winter and Milstein, 1991). These antibodies can also be humanized by known methods to avoid immune reactivity when used in therapeutic methods (Breedveld, 2000).

The antibodies to Region 2 or Region 3 can be raised against the whole factor VIII or IX, after which antibodies can be selected by routine methods (e.g., by ELISA with monoclonal antibodies, or affinity purification with polyclonal antibodies) for binding to Region 2 or Region 3 by, e.g., determining whether the antibody binds to the polypeptide agents previously disclosed that are homologous to these regions in factor VIII or factor IX. Alternatively, the antibodies to Region 2 or Region 3 can be raised against the previously disclosed polypeptides themselves. As is well known, antibodies can be raised against a short polypeptide by conjugating the peptide to an immunogenic carrier molecule such as bovine serum albumin or keyhole limpet hemocyanin. Under these conditions, antibodies will be produced against the carrier molecule as well as the polypeptide. The antibodies to the polypeptide can then be selected by routine methods.

The utility of any particular agent in inhibiting the interaction of factor VIIIa and IXa can also be ascertained by evaluating the binding of the polypeptide or derivative to the factor VIIIa or IXa by any of a number of methods that are well known in the art. For example, the polypeptide or derivative can be labeled with a radioactive agent or a dye such as a fluorescent dye, and unbound vs. bound polypeptide or derivative can be determined by methods such as chromatography or electrophoresis, where the chromatographic or electrophoretic conditions are selected where unbound polypeptide migrates differently than polypeptide bound to factor VIIIa or factor IXa. Alternatively, bound vs. unbound polypeptide or derivative can be determined by dialysis, using a membrane which allows the passage of unbound labeled polypeptide or derivative but not polypeptide or derivative bound to factor VIIIa or factor IXa. Another alternative method for determining polypeptide or derivative bound to factor VIIIa or factor IXa is by the determination of displacement of labeled polypeptide from factor VIIIa or factor IXa that is adsorbed to a solid phase.

The inhibitory agents disclosed above can be supplied as a polynucleotide that encodes the agent, wherein the polynucleotide is operably linked to a control sequence that allow the polynucleotide to be translated in a mammalian cell. These agents are useful, e.g., in gene therapy applications. Gene therapy reagents and control sequences for cardiovascular and hematology applications are well known in the art. See, e.g., Carmeliet and Collen, 1996; Clowes, 1997; Schwartz and Moawad, 1997; and Yla-Herttuala and Martin, 2000.

As used herein, “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences.

In other embodiments, the present invention is directed to a composition that induces coagulation. The composition comprises the portions of the amino acid sequence of factor VIIIa that interact with factor IXa, or derivatives thereof. The composition could comprise the entire portion of the factor VIIIa amino acid sequence that encompasses the factor IXa-interacting portions (for example E440-K713 of SEQ ID NO:1), or it could comprise the amino acid fragments that interact with factor IXa connected by linkers designed to align the interacting portions to the proper areas of factor IXa.

It is contemplated that the polypeptides or derivatives of the present invention are usually employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically acceptable excipients for modifying or maintaining release or absorption or penetration across the blood-brain barrier. Such excipients are those substances usually and customarily employed to formulate dosages for parenteral administration in either unit dosage or multi-dose form or for direct infusion by continuous or periodic infusion.

It is also contemplated that certain formulations comprising the polypeptides or derivatives are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic and nucleic acid degradation and/or substances that promote absorption such as, for example, surface active agents.

In other embodiments, the present invention is directed to a method of treatment to prevent coagulation in a patient in need thereof. The method comprises administering at least one of the agents described above in a pharmaceutically acceptable excipient. In preferred embodiments, the patient is a human, but the method could easily be adapted to any other vertebrate subject. In doing so, the non-human vertebrate Region 2 and Region 3 analogous to human Region 2 and Region 3 can be routinely identified by the modeling methods disclosed herein.

This method is useful for any disorder where inhibition of coagulation is desired. Preferred disorders are cardiovascular disorders involving inappropriate coagulation. Examples include thrombosis, atherosclerosis and restenosis. Thrombosis is defined herein as the formation, development, or presence of a blood clot in a blood vessel or the heart, including where cerebral vessels are involved, leading to stroke. Thrombosis can be usefully treated by this method to prevent further clot formation.

Atherosclerosis is useful for treatment by these methods, since clot formation, induced by tissue factor in a ruptured or fissured atherosclerotic plaque, often induces unstable angina and myocardial infarction (Toschi et al., 1997; Ardissino et al., 2000). Thus, treatment of an atherosclerosis patient with the agents disclosed above would prevent such clot formation. Similarly, restenosis, defined herein as a reformation of an occlusion in a blood vessel after an occlusion has been corrected, e.g., with angioplasty, often occurs due to tissue factor induced coagulation (Oltrona et al., 1997; Gallo et al., 1998). Thus, treatment of a patient in danger of restenosis would prevent such occlusions from occurring.

In these embodiments, the agent can be administered by any method known in the art that is capable of providing the agent to the bloodstream where clotting inhibition is desired. Intravenous administration is preferred, since that introduces the agent directly into the bloodstream. The agent can also be administered in the form of a polynucleotide encoding the agent, wherein the polynucleotide is operably linked to a control sequence that allows the polynucleotide to be translated in cells into which the polynucleotide is introduced. This gene therapy approach can also involve ex vivo introduction of the polynucleotide-control sequence combination into a cell such as a lymphocyte or macrophage, which is then transferred to the patient, where the polynucleotide is expressed and the agent is produced. For general reviews of applicable gene therapy approaches, see Carmeliet and Collen, 1996; Clowes, 1997; Schwartz and Moawad, 1997; and/or Yla-Herttuala and Martin, 2000.

The present invention is also directed to a method of identifying a compound having anti-coagulation activity. The method comprises combining the test compound with reagents that exhibit Region 2 or Region 3 interaction, then determining whether the compound displaces that interaction. For example, the compound could be combined with factor IXa and an agent that interacts with factor IXa at Region 2 or Region 3 (e.g., a peptide comprising E445 to K570 of factor VIIIa). If the compound disrupts the interaction, it would likely be a compound that would inhibit coagulation. Any of several means known in the art can be utilized to determine whether the compound displaces the agent. Preferably, that determination is made by evaluating whether the compound prevents the binding of the agent to factor VIIIa or factor IXa, wherein the agent is labeled with a detectable marker. The detectable marker can be any of a number of well-known markers, including fluorescent markers, radioactive markers, and spin labels. In preferred embodiments of this method, the agent selected should not bind with a high affinity to factor VIIIa or factor IXa at Region 2 or Region 3, since the binding of an agent with high affinity would be difficult to displace with the test compound, and test compounds that might otherwise be effective in preventing the interaction of factor VIIIa with factor IXa would not be able to displace the agent.

In additional embodiments, the present invention is directed to a method of preventing coagulation in a blood sample. The method comprises adding an agent as previously described to the sample. These agents can be homologous to factor VIII or factor IX at Region 2 or Region 3. Alternatively, the agents can be non-homologous to factor VIII or factor IX at Region 2 or Region 3. As previously discussed, the latter agent can, for example, comprise an antibody binding site or a polypeptide that does not comprise an antibody binding site.

In these methods, the polypeptide or derivative can be added to the blood sample as a liquid or dried preparation. Alternatively, the polypeptide can be present in the container that receives the blood sample (for example a vacutainer), in order for the blood sample to be exposed to the polypeptide when the sample enters the container. The quantity of the polypeptide or derivative added to the container can be determined without undue experimentation, merely by determining the quantity of the polypeptide or derivative necessary to prevent coagulation of the quantity of blood which is to be drawn in the sample.

Industrial Application

The compositions and methods of the present invention provide novel treatments to prevent coagulation in vivo, methods for preventing coagulation in blood samples, and methods for identifying agents that have anti-coagulation activity.

Preferred embodiments of the invention are described in the following example. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims which follow the examples.

The procedures disclosed herein which involve the molecular manipulation of nucleic acids are known to those skilled in the art. See generally Fredrick M. Ausubel et al. (1995), “Short Protocols in Molecular Biology”, John Wiley and Sons, and Joseph Sambrook et al. (1989), “Molecular Cloning, A Laboratory Manual”, second ed., Cold Spring Harbor Laboratory Press, which are both incorporated by reference.

### EXAMPLE 1

This example provides evidence demonstrating the importance of the interaction of the A2 subunit of Factor VIIIa with Factor IXa at Region 1, Region 2, and Region 3.

Experimental Procedures

Reagents.

Benzoyl-Ile-Glu-Gly-Arg-p-nitroanilide (S-2222) was purchased from Helena Laboratories. Dansyl-Glu-Gly-Arg-chloromethyl ketone (dEGR-ck) was obtained from Calbiochem. Phosphatidylcholine (PC), phosphatidylserine (PS), phosphatidylethanolamine (PE), recombinant hirudin, and fatty acid-free bovine serum albumin (BSA) were obtained from Sigma. Phospholipid (PL) vesicles containing 75% PC and 25% PS were prepared by the method of Husten et al., 1987. For fluorescence experiments, phospholipid vesicles were comprised of 20% PS, 40% PC and 40% PE and were prepared using octyl glucoside as previously described (Mimms et al., 1981). Recombinant factor VIII preparations (Kogenate®) were a gift from Drs. Lisa Regan and Jim Brown of Bayer Corporation. Purified recombinant factor VIII was also a generous gift from Debra Pittman of the Genetics Institute. Normal plasma factor IX (IX_{NP}) and factor X was isolated as previously described (Bajaj and Birktoft, 1993) and factor Xa was prepared as outlined in Bajaj et al., 1981. Purified human factor XIa and thrombin were purchased from Enzyme Research Laboratories (South Bend, Ind.). A synthetic peptide corresponding to the A2 subunit residues 558-565 (Ser-Val-Asp-Gln-Arg-Gly-Asn-Gln) (SEQ ID NO:17) was obtained as described in in Fay and Koshibu, 1998, and its concentration was determined by amino acid analysis.

Proteins.

The Kogenate® concentrate was fractionated to separate factor VIII from albumin following S-Sepharose chromatography as outlined in Fay et al., 1993. Factor VIIIa was prepared from factor VIII using thrombin and subsequently purified using CM-Sepharose chromatography (Curtis et al., 1994), and the A2 subunit and A1/A3-C1-C2 dimer were separated by Mono S chromatography (Fay et al., 1991a). The A2 subunit was further purified using an anti-A2 immunoaffinity column (Fay et al., 1991a). The purified A2 subunit was essentially homogeneous (>95% pure) as judged by SDS-PAGE. For some experiments, proteins were concentrated using a MicronCon concentrator (Millipore, 10 kDa cut-off). The concentration of the A2 subunit was determined by the coomassie blue dye binding method of Bradford (1976). Wild-type factor IX (IX_{WT}), as well as mutants IX_{R333Q} (a point mutant in which Arg333 is replaced by Gln), IX_{VIIhelix} (a replacement mutant in which helix 330-338 (c162-170) is replaced by that of factor VII) and IX_{PCEGF1} (a replacement mutant in which EGF1 domain is replaced by that of protein C) were constructed, expressed and purified as described earlier (Mather and Bajaj, 1999; Zhong et al., 1994; Zhong and Bajaj, 1993). Purified proteins were homogeneous on SDS-PAGE and contained normal Gla content (Mather and Bajaj, 1999; Zhong et al., 1994).

Preparation of dEGR-ck Inhibited Factor IXa Proteins.

Each factor IX protein was activated at 200 μg/ml by factor XIa (2 μg/ml) for 90 min. The buffer used was TBS, pH 7.5 (50 mM Tris, 5 mM NaCl, pH 7.5) containing 5 mM CaCl₂. SDS-PAGE analysis revealed full activation of each factor to factor IX to factor IXa without degradation in the autolysis loop (Mather et al., 1997). dEGR-IXa_{WT}, and various dEGR-IXa mutant proteins free of dEGR-ck were obtained as described previously (Mather et al., 1997).

Activation of Factor X by Each Factor IXa Protein in the Presence of Only Ca²⁺ and PL.

For these studies, each factor IX was activated with factor XIa/Ca²⁺ as described above. For factor X activation studies, the concentration of factor IXa was kept at 20 nM and the buffer used was TBS/BSA (TBS with 200 mg/ml BSA) containing 5 mM CaCl₂. The concentrations of PL used were 10, 25, 50, and 100 μM in different sets of experiments. The concentration of factor X at each PL concentration ranged from 25 nM to 3 μM. The activations were carried out for 5-15 min and the amount of factor Xa generated was measured by hydrolysis of S-2222 as described previously (Mather and Bajaj, 1999; Mather et al., 1997). The Km and kcat values were obtained using the program GraFit from Erithacus Software.

Determination of EC₅₀ of Interaction of Factor IXa Proteins with A2 Subunit.

The EC₅₀ (functional Kd) of binding of each factor IXa protein with A2 subunit was measured essentially as described previously for its interaction with intact factor VIIIa (Mather and Bajaj, 1999; Mather et al., 1997). For these experiments, concentrations of factor IXa and factor X were kept constant, and the rates of formation of factor Xa were determined at increasing concentrations of A2 subunit. Reaction mixtures contained 5 nM IXa protein, 250 nM factor X, 25 μM PL and various concentrations of A2 subunit in TBS/BSA, pH 7.5 containing 5 mM CaCl₂. Reactions were carried out at 37° C. for 5-20 minutes and stopped by adding 1 μL of 500 mM EDTA. The amount of factor Xa generated was determined by S-2222 hydrolysis as described previously (Mather and Bajaj, 1999; Mather et al., 1997). The EC₅₀ was obtained by fitting the data to a single site ligand binding equation (Eq. 1) by non-linear regression analysis using the program GraFit from Erithacus Software.

$$V = \frac{{V}_{max} L}{{EC}_{50} + L} \qquad ( Eq . 1 )$$

Where V is the rate of formation of factor Xa at a given concentration of the A2 subunit, denoted by L, and Vₘₐₓ is the rate of factor Xa formation by the factor IXa:A2 subunit complex. EC₅₀ is the functional Kd defined as the concentration of free A2 subunit yielding 50% of the Vₘₐₓ. The background rate of factor Xa generation was obtained by carrying out the reaction in the absence of the A2 subunit. This represented less than 1% of the Vₘₐₓ and was subtracted before data analysis. To obtain EC₅₀ values as a function of substrate concentration, a series of experiments were performed in which factor X was varied from 50 nM to 1 μM.

Determination of Kd_{A2} of Binding of dEGR-IXa Proteins to A2 Subunit.

The apparent Kd (termed Kd_{A2}) for binding of each dEGR-IXa protein to the A2 subunit was determined by its ability to inhibit factor IXa_{WT}:A2 subunit interaction in the tenase complex as described earlier for intact factor VIIIa (Mather and Bajaj, 1999; Mather et al., 1997). The reactions were carried out as described for the EC₅₀ experiments above except dEGR-IXa and IXa_{WT} were mixed prior to addition of the A2 subunit; this ensured steady state conditions. Mixtures contained 100 nM IXa_{WT}, 30 nM A2 subunit, 250 nM factor X, 25 μM PL, and various concentrations of dEGR-IXa proteins in TBS/BSA, pH 7.5 containing 5 mM CaCl₂. The IC₅₀ (concentration of inhibitor required for 50% inhibition) was determined by fitting the data to the IC₅₀ four-parameter logistic equation of Halfman (1981) given below (Eq. 2).

$$y = \frac{a}{1 + {( x / {IC}_{50} )}^{s}} + background \qquad ( Eq . 2 )$$

where y is the rate of Xa formation in the presence of a given concentration of dEGR-IXa protein represented by x, a is the maximum rate of factor Xa formation in the absence of dEGR-IXa, and s is the slope factor. Each point was weighted equally, and the data were fitted to Equation 2 using the nonlinear regression analysis program GraFit from Erithacus Software. The background value represented ˜5% of the maximum rate of Xa formation in the absence of dEGR-IXa. To obtain Kd_{A2} values for the interaction of dEGR-IXa proteins with A2, we used the following equation (Eq. 3) as described by Cheng and Prusoff (1973) and further elaborated by Craig (1993).

$${Kd}_{A2} = \frac{{IC}_{50}}{1 + ( A / {EC}_{50} )} \qquad ( Eq . 3 )$$

where A is the concentration of IXa_{WT}, and EC₅₀ is the concentration of factor IXa_{WT} that gives a 50% maximum response in the absence of the competitor at a specified concentration of factor X used in the experiment.

Fluorescence Quenching of the Dansyl Moiety in dEGR-IXa by the A2 Subunit.

The effect of the A2 subunit on the emission intensity of the dansyl moiety in each dEGR-IXa protein was determined using the SLM AB2 spectrofluorometer. Each reaction mixture contained 220 nM dEGR-IXa in 20 mM Hepes, pH 7.2, 100 mM NaCl, 5 mM CaCl₂, 0.01% Tween, 200 μg/ml BSA and 100 μM PL vesicles. The excitation wavelength was 340 nm (slit width, 8 nm) and the emission wavelength was 540 nm (slit width; 8 nm). First, blank values (in triplicate) were obtained for the buffer containing PL. dEGR-IXa was then added and the emission intensity in the absence of the A2 subunit was recorded. Each reaction mixture was subsequently titrated with the A2 subunit and the emission readings (in triplicate) were obtained at each time point. The fluorescence emission intensity at each point was corrected for increases in the reaction volume prior to analysis of the data. The volume of added A2 subunit did not exceed 10% of the total volume. Data are presented as F/F₀ where F₀ is the emission intensity in the absence of A2 subunit and F is the intensity at a given A2 subunit concentration.

Determination of the Apparent Kd_{peptide} of Binding of Each Factor IXa to the A2 558-565 Peptide.

The apparent Kd (termed Kd_{peptide}) for binding of each factor IXa to the A2 558-565 peptide was determined by its ability to inhibit the respective IXa:A2 subunit interaction as measured by reduction in the rate of factor X activation in the tenase system. The reaction mixtures for both IXa_{WT} and IXa_{PCEGF1} contained 100 nM factor IXa, 30 nM A2 subunit, 250 nM factor X, and 25 μM PL in TBS/BSA, pH 7.5 with 5 mM CaCl₂. The reaction mixture for IXa_{R333Q} contained 300 nM factor IXa instead of 100 nM used for IXa_{WT} or IXa_{PCEGF1}; concentrations of other components were the same. The amount of factor Xa generated was determined by hydrolysis of S-2222. The IC₅₀ values were obtained using Eq. 2. Here, y is the rate of factor Xa formation in the presence of a given concentration of the A2 558-565 peptide represented by x, and a is the maximum rate of factor Xa formation in the absence of the A2 peptide. Eq. 3 was then used to obtain the apparent Kd_{peptide} values. In this context, A is the concentration of IXa_{WT}, IXa_{PCEGF1}, or IXa_{R333Q}, and EC₅₀ is the apparent Kd_{A2} for the respective IXa:A2 subunit interaction.

Molecular Modeling.

The three (A1, A2, and A3) domains in factor VIIIa are homologous to the three respective domains in ceruloplasmin (Pemberton et al., 1997; Church et al., 1984). The A1, A2, and A3 domains in factor VIIIa were modeled using the coordinates of each respective domain of ceruloplasmin (Zaitseva et al., 1996). Each domain was modeled using the homology model building module from Biosym/MSI, San Diego, Calif. as well as the Swiss-Model server using the optimize mode (Peitsch, 1996; Guex et al., 1999). The two approaches used in building the homology models resulted in minor differences between the structure of each of the A subunits. However, the structure pertaining to the loop containing the 3₁₀ helical turn involving residues 558-565 (Region 1) as well as the other interface regions of the A2 subunit implicated in binding to factor IXa (Regions 2 and 3) were invariant between the two models. Further, the Biosym/MSA models of all three A subunits were similar to those published earlier by Pemberton et al. (1997). Thus, we used the coordinates of Pemberton et al. (1997; also given at the Hemostasis Research Group web site, http://europium.csc.mrc.ac.uk/usr/WWW/WebPages/main.dir/main.htm) in building the interface between the A2 subunit and the protease domain of factor IXa. Details are provided in the Results and Discussion section.

Results and Discussion

Activation of Factor X by Various Factor IXa Proteins in the Presence of Only Ca²⁺ and PL.

The kinetic constants for the activation of factor X were obtained by various factor IXa proteins in the presence of Ca²⁺ and several concentrations of PL. This analysis was performed to establish whether or not the factor IXa proteins under investigation bind to Ca²⁺ and PL normally and possess a functional active site. The kinetic constants obtained in the absence of factor VIIIa are listed in Table 2. All mutants activated factor X normally and the specificity constant (kcat/Km) for each mutant at different PL concentrations did not differ appreciably from that observed for IXa_{WT} or IXa_{NP}. The increase in Km values at higher concentrations of PL for WT or for a given mutant may reflect binding of factor IXa and factor X to different PL vesicles (Mann et al., 1988; van Dieijen et al., 1981). Further, our Km and kcat values are in close agreement with the earlier published data (Fay and Koshibu, 1998; van Dieijen et al. 1981). Consistent with earlier observations (van Dieijen et al. 1981), we also observed a slight increase in kcat for each factor IXa protein at higher concentrations of PL. Cumulatively, our data presented in Table 2 indicate that the factor IXa mutants under investigation interact with Ca⁺² and PL normally. Further, in the absence of factor VIIIa, activation of factor X by these IXa mutants is not impaired.

TABLE 2
Kinetic parameters of factor X activation in the absence of factor
VIIIa. The concentration of each reagent in the reaction mixture
was: 20 nM factor IXa, 5 mM CaCl₂, and varying
concentrations of factor X ranging from 25 nM to 3 μM.

| Protein | PL (μM) | Km (μM) | kcat (min⁻¹) | kcat/Km (μM⁻¹ min⁻¹) |
|---|---|---|---|---|
| IXa_{NP} | 10 | 0.10 | 0.012 | 0.108 |
| | 25 | 0.16 | 0.022 | 0.138 |
| | 50 | 0.21 | 0.032 | 0.156 |
| | 100 | 0.63 | 0.062 | 0.098 |
| IXa_{WT} | 10 | 0.09 | 0.010 | 0.110 |
| | 25 | 0.12 | 0.012 | 0.102 |
| | 50 | 0.24 | 0.024 | 0.100 |
| | 100 | 0.57 | 0.038 | 0.066 |
| IXa_{PCEGF1} | 10 | 0.13 | 0.010 | 0.076 |
| | 25 | 0.23 | 0.018 | 0.080 |
| | 50 | 0.26 | 0.028 | 0.110 |
| | 100 | 0.54 | 0.038 | 0.070 |
| IXa_{R333Q} | 10 | 0.11 | 0.012 | 0.108 |
| | 25 | 0.18 | 0.018 | 0.100 |
| | 50 | 0.23 | 0.030 | 0.130 |
| | 100 | 0.61 | 0.040 | 0.066 |
| IXa_{VIIhelix} | 10 | 0.12 | 0.014 | 0.116 |
| | 25 | 0.20 | 0.020 | 0.100 |
| | 50 | 0.25 | 0.028 | 0.112 |
| | 100 | 0.55 | 0.042 | 0.076 |

A42 Subunit Mediated Enhancement of Factor X Activation by Various IXa Mutants.

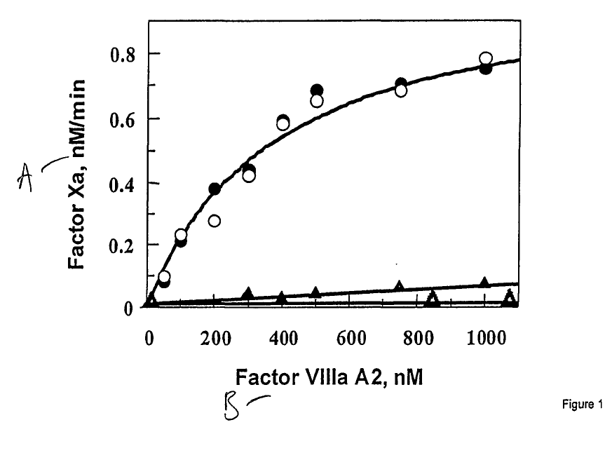

In this section, we evaluated the ability of the A2 subunit to augment factor X activation by various factor IXa mutants. These data are presented in FIG. 1. The presence of the A2 subunit in the reaction mixtures enhanced the factor X-activating activity of IXa_{PCEGF1} to the same extent as that of IXa_{WT}. However, the ability of the A2 subunit to potentiate the activity of IXa_{R333Q} was severely impaired and it was absent for the IXa_{VIIhelix}. Next, we determined the EC₅₀ (functional Kd) values for interaction of each factor IXa protein with the A2 subunit using Eq. 1. Fitting the data to a single site-binding model yielded an apparent Kd of 257±31 for both IXa_{WT} and IXa_{PCEGF1}; for IXa_{R333Q} or IXa_{VIIhelix}, it could not be calculated. These data strongly indicate that the helix 330 (c162) of factor IXa interacts with the A2 subunit of VIIIa.

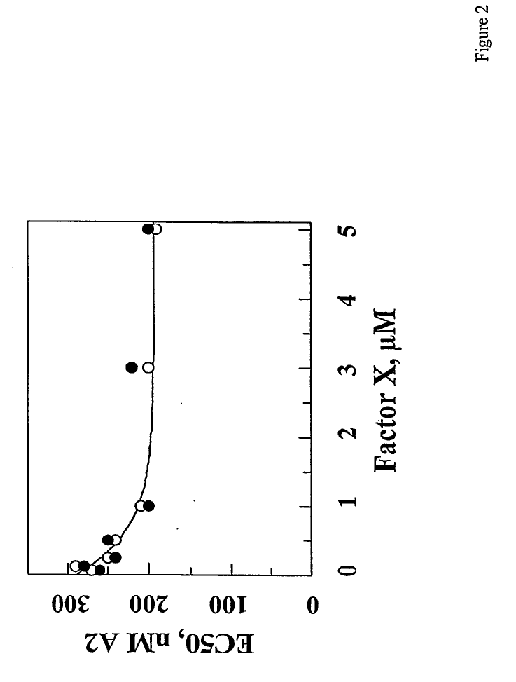

In further experiments, we measured the EC₅₀ values for interaction of IXa_{WT} and of IXa_{PCEGF1} with the A2 subunit using different concentrations of factor X ranging from 50 nM to 5 μM. These data are presented in FIG. 2. At each concentration of factor X, the concentration of IXa was held constant at 5 nM and the rate of factor Xa generation was determined in the presence of increasing concentrations of the A2 subunit. The EC₅₀ values ranged from ˜300 nM at lower concentrations of factor X (<150 nM) to ˜200 nM at higher concentrations of factor X (>1 μM) for both IXa_{WT} and IXa_{PCEGF1}. Our functional Kd (EC₅₀) values ranging from 200-300 nM for the interaction of IXa_{WT} (or IXa_{PCEGF1}) and the A2 subunit employing different factor X concentrations are consistent with the EC₅₀ values obtained earlier using similar conditions for IXa_{NP} and the A2 subunit (Fay and Koshibu, 1998). From these observations, we conclude that factor X does not appreciably influence the functional Kd of IXa:A2 subunit interaction. This is in contrast to the results obtained using factor VIIIa where factor X reduces the functional Kd of IXa:VIIIa interaction by ˜10-fold (Mather et al., 1997). More importantly, our data with the IXa_{PCEGF1} mutant indicate that the EGF1 domain of factor IXa does not interact with the A2 subunit of factor VIIIa.

Determination of Apparent Kd_{A2} Values for the Interaction of A2 Subunit with dEGR-IXa Proteins.

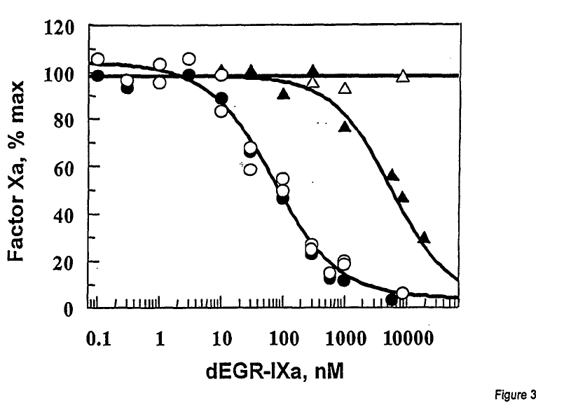

Here, we investigated the steady state inhibition of IXa_{WT}:A2 interaction by different dEGR-IXa proteins. These data are presented in FIG. 3. The IC₅₀ values were obtained using Equation 2 and the respective apparent Kd_{A2} values were obtained using Equation 3. dEGR-IXa_{WT} and dEGR-IXa_{PCEGF1} interacted with the A2 subunit with a similar Kd_{A2} of ˜100 nM, whereas dEGR-IXa_{R333Q} interacted with the A2 subunit with a Kd_{A2} of ˜1.8 μM and dEGR-IXa_{VIIhelix} failed to compete with IXa_{WT} up to 12 μM concentration. The apparent Kd_{A2} (˜100 nM) obtained from the inhibition data (FIG. 3) and EC₅₀ values (˜250 nM) obtained from the potentiation of factor X activation data (FIGS. 1 and 2) for the factor IXa_{WT} and IXa_{PCEGF1} are in close agreement with each other. Of significance is the observation that the mutations in the helix 330 (c162) of the protease domain of factor IXa severely impairs its interaction with the A2 subunit.

Effects of A2 subunit on the Fluorescence Emission of dEGR-IXa Proteins.

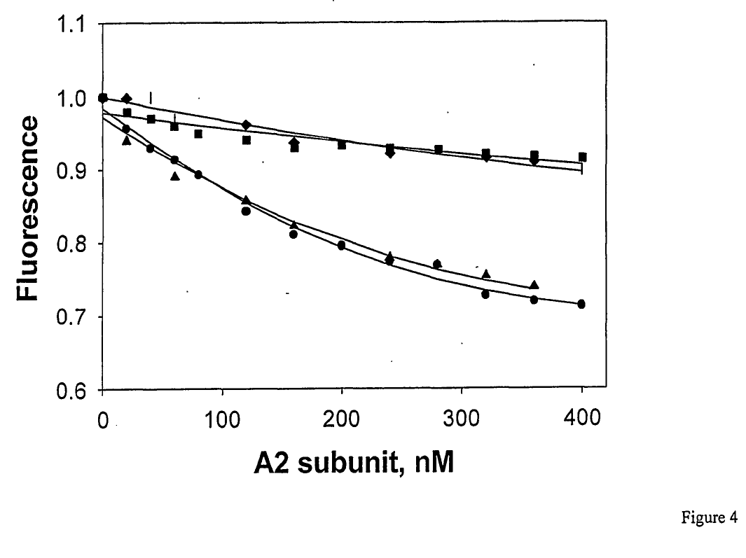

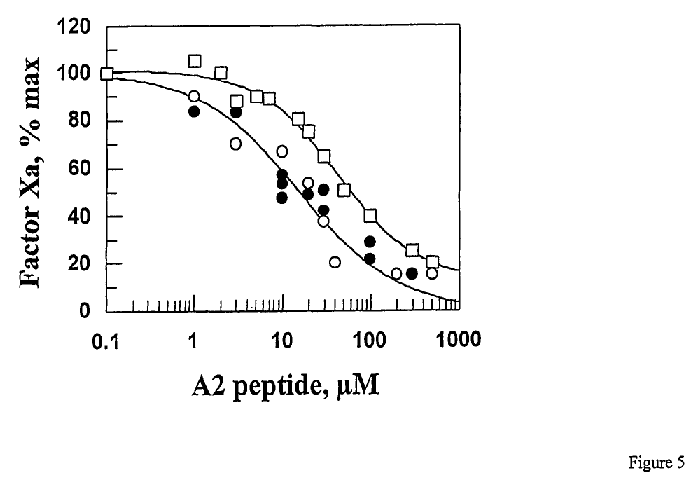

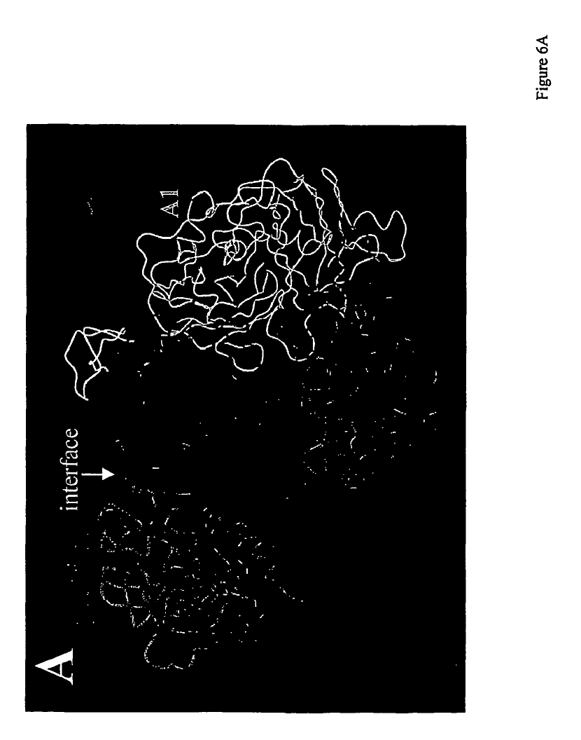

Since dansyl emission is quite sensitive to its environment, we examined the changes in dansyl emission intensity (excitation wavelength, 340 nm and emission wavelength, 540 nm) of dEGR-IXa proteins in the presence of increasing concentrations of the A2 subunit. Reaction mixtures contained 220 nM of each dEGR-IXa protein, 100 μM PL and various concentrations of the isolated A2 subunit. The results are presented in FIG. 4. For IXa_{WT} and IXa_{PCEGF1}, a dose-dependent decrease in the fluorescence emission of the dansyl probe was observed. However, little if any change in the emission intensity was observed when the A2 subunit was titrated into the reaction mixtures containing factor IXa_{R333Q} or IXa_{VIIhelix}. A nonlinear least squares fitting to the data for IXa_{WT} or IXa_{PCEGF1} to a bimolecular association model yielded a plateau value of 0.59±0.05 for F/F₀ and an apparent Kd_{A2} value of ˜100 nM for each protein. These results suggest that the isolated A2 subunit interacts equivalently with these two forms of factor IXa, similarly modulating the emission of the active site-labeled dansyl probe. The apparent Kd_{A2} value of ˜100 nM for factor IXa_{WT} or IXa_{PCEGF1} obtained using the fluorescence quenching measurements is in agreement with the values obtained from steady state experiments. Consistent with the data presented in FIGS. 1 and 3, these fluorescence results suggest that the factor IXa_{R333Q} and IXa_{VIIhelix} mutants are severely impaired in their interactions with the A2 subunit.

Determination of Apparent Kd_{peptide} Values for Binding of Factor IXa Proteins to the A2 558-565 Peptide.

The data presented thus far strongly indicate that the A2 subunit interacts with the residues of the helix 330 (c162) of factor IXa. Previous studies also suggest that residues 558-565 of the A2 subunit are involved in binding to factor IXa (Fay and Koshibu, 1998). However, it is not known whether the 558-565 peptide region of the A2 subunit represents the site of direct interaction with the helix 330 of factor IXa. We investigated this possibility by measuring the affinity of the A2 558-565 peptide for IXa_{WT}, IXa_{PCEGF1}, and IXa_{R333Q}. These data are presented in FIG. 5. The A2 558-565 peptide inhibits the interaction of IXa_{WT} and IXa_{PCEGF1} with similar IC₅₀ values of ˜8 μM. The present IC₅₀ value [˜8 μM] for the A2 peptide inhibition of the IXa_{WT}:A2 subunit interaction is five-fold lower than the IC₅₀ value [˜40 μM] obtained from the inhibition studies of the A2 subunit enhancement of IXa_{NP} activity [Fay and Koshibu, 1998]. The difference in IC₅₀ values is most likely due to the different concentrations (30 nM in the present study vs. 240 nM in the previous study) of the A2 subunits used in the two studies. However, the A2 558-565 peptide inhibited the IXa_{R333Q}:A2 subunit interaction with an IC₅₀ value of ˜70 μM, which is ˜9-fold higher than the value obtained for IXa_{WT} or IXa_{PCEGF1} (FIG. 5). The Cheng and Prusoff relationship (Cheng and Prusoff, 1973; Craig, 1993) was then used to obtain apparent Kd_{peptide} values for each factor IXa protein. These apparent Kd_{peptide} values along with the changes in Gibbs free energy are listed in Table 3. Notably, the increase in apparent Kd_{A2} or Kd_{peptide} for IXa_{R333Q} is similar (˜15-fold) as compared to the apparent Kd_{A2} or Kd_{peptide} obtained for IXa_{WT} (or IXa_{PCEGF1}). Further, the difference in ΔG° for the interaction of the A2 subunit with IXa_{WT} (or IXa_{PCEGF1}) and IXa_{R333Q} is 1.72 kcal mol⁻¹. This difference in ΔG° is essentially the same as that (1.62 kcal mol⁻¹) obtained for the interaction of A2 peptide with IXa_{WT} (or IXa_{PCEGF1}) and IXa_{R333Q}. If the A2 558-565 peptide bound to a different region than the helix 330 of factor IXa, then one would expect it to bind to IXa_{R333Q} with the same affinity as that for IXa_{WT}. Since this is not the case, our data support a conclusion that the helix 330 (c162) in factor IXa is most likely in direct contact with the 558-565 region of the A2 subunit.

TABLE 3
Apparent Kd and Gibbs free energy values for the interaction of
various factor IXa proteins with the A2 subunit and the A2 558-565
peptide. Apparent Kd_{A2} values are from FIG. 3 and apparent
Kd_{peptide} values are from FIG. 5.

| | App | ΔG°_{A2}b | App | ΔG°_{peptide}b |
|---|---|---|---|---|
| Protein | Kd_{A2} − nM | kcal mol⁻¹ | Kd_{peptide} − μM | kcal mol⁻¹ |
| IXa_{WT} | 100 ± 11 | 9.54 | 4 ± 1 | 7.36 |
| IXa_{PCEGF1} | 114 ± 15(1)^{a} | 9.47(0.07)^{c} | 4 ± (1)^{a} | 7.36(0.00)^{c} |
| IXa_{R333Q} | 1850 ± 82(18) | 7.82(1.72) | 62 ± 9(15) | 5.74(1.62) |
| IXa_{VIIhelix} | >10³ | ND^{d} | ND | ND |

^{a}The fold-change in apparent Kd values (mutant/WT) is given in parentheses.
^{b}Gibbs free energy values were calculated using the equation, ΔG° = RTlnKd, where R is the gas constant (1.987 × 10⁻³ kcal mol⁻¹ deg⁻¹), T is the absolute temperature (298 Kelvin), and Kd is the dissociation constant.
^{c}The change in ΔG° values between the mutant and WT is given in parentheses.
^{d}ND, not determined

Modeling of the Interface Between the Protease Domain of Factor IXa and the A2 Subunit of Factor VIIIa.

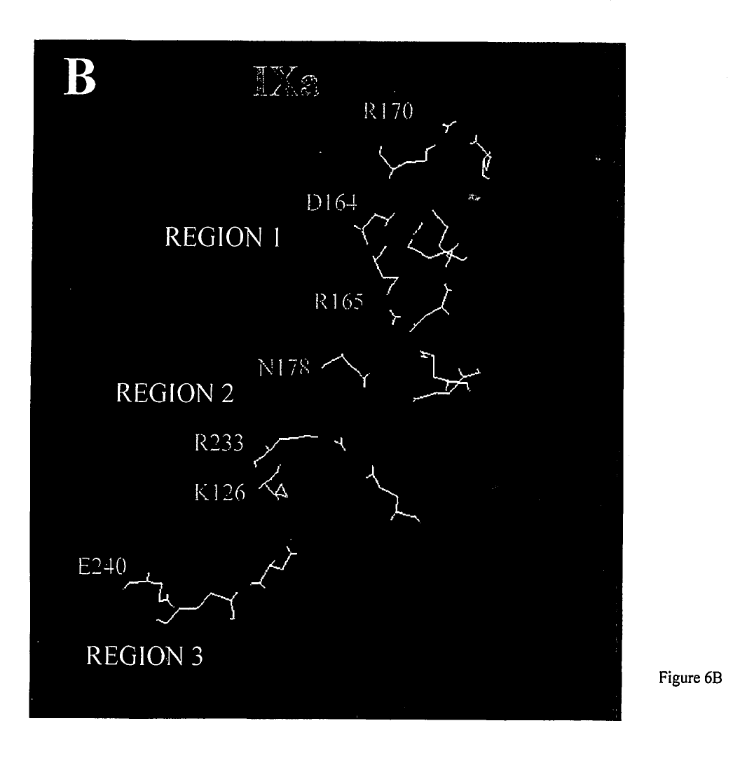

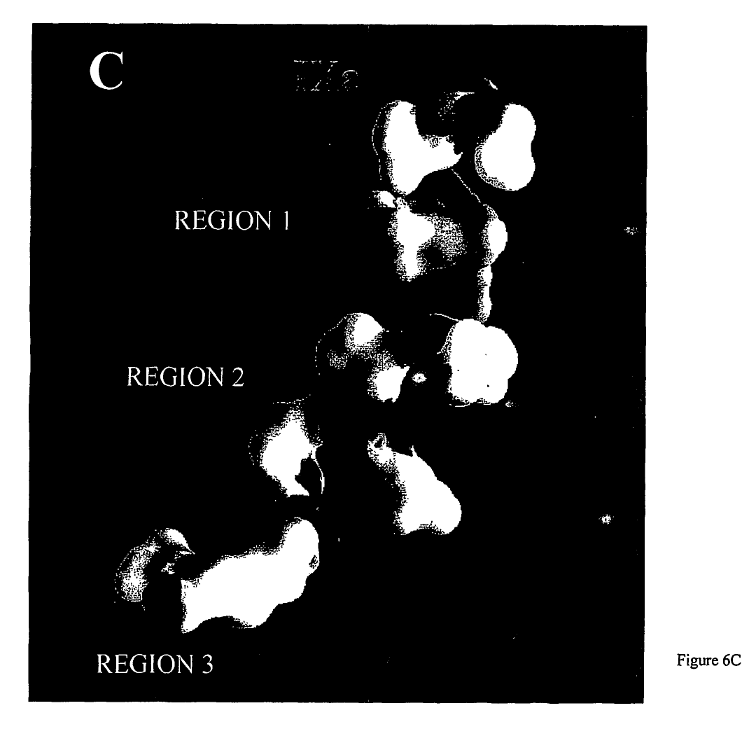

Based upon the preceding information, we modeled the interface between the protease domain of factor IXa (Hopfner et al., 1999; PDB code 1RFN) and the A2 subunit (see Experimental Procedures) by bringing together the helix 330 of factor IXa and the 3₁₀ helical turn in residues 558-565 of the A2 subunit and maximizing the interaction among the charged residues. Emphasis was also given for interactions involving hydrogen bonds and hydrophobic contacts. An important guiding principle in the construction of this interface model was that the Gla domain of factor IXa and the C2 domain of factor VIIIa must be oriented such that each may contact the PL surface. To achieve this, the A2 structure (along with the A1 and A3 subunits) was rotated and translated as a rigid body. The principal approach used was that described earlier by Tulinsky and coworkers in building the prothrombin model from the structures of fragment 1 and prethrombin (Arni et al, 1994). Minor adjustments in the side chains of both proteins were also made. All residues in the interface of both proteins were checked for distances to insure no improper contacts (Laskowski et al., 1993). The interface model that resulted from this approach is shown in FIG. 6A. In this display, the Gla domain of factor IXa and the C2 domain of factor VIIIa are projecting away from the viewer.

In addition to the A2 558-565 region and the factor IXa 330-338 region, two other regions that apparently also play a role in the interaction of A2 subunit and the protease domain (Region 2 and Region 3) were identified. The details of the three interface regions are shown in FIG. 6B. It appears that electrostatic forces might play a significant role in the interaction between the A2 subunit and the protease domain, and an electrostatic potential for the interface calculated using the program GRASP (Arni et al., 1994) is shown in FIG. 6C. Further, in addition to the electrostatic interactions outlined in FIG. 6, hydrophobic and polar uncharged interactions between T343 (c175) and Y345 (c1 77) of factor IXa and H444 of the A2 subunit were observed. Moreover, a hydrogen bond between N258 (c93) of factor IXa and S709 of the A2 subunit could also be formed. Importantly, a significant hydrophobic patch involving I566 and M567 in the A2 subunit and I298 (c129B), Y295 (c128), F299 (c130), F302 (c133), F378 (c208), and F98 (EGF2 domain) in factor IXa was noted. Thus, it appears that the hydrogen bonds as well as the hydrophobic and electrostatic interactions all play important roles in the interface between factor IXa and the A2 subunit. In this context, an apparent Kd_{A2} of ˜100 nM observed for this interaction reflects the net change in free energy involved in making and breaking such bonds.

A factor IXa-interactive site comprised of residues 484-509 in the A2 subunit that was identified using a monoclonal antibody (Fay and Scandella, 1999) does not appear to contact the protease domain in the interface model. However, it should be noted that in a previous study, Lollar et al. (1994) concluded that this same monoclonal antibody does not interfere with the IXa:VIIIa interaction. The reason(s) for the differing results obtained in the two studies (Fay and Scandella, 1999 and Lollar et al., 1994) is not fully understood. Further in the interface model shown in FIG. 6A, the 484-509 region in the A2 subunit is not in close proximity to the 558-565 interface region. Therefore it is likely that this monoclonal antibody prevents the association of the A2 subunit with factor IXa through steric interference.

Analysis of Hemophilia Databases.

Of significance is the observation that numerous mutations in the helix 330 (c162) of factor IXa cause hemophilia B (Green et al., 2000; Mathur and Bajaj, 1999) while several mutations in or near factor VIII residues 558-565 result in hemophilia A (Hemostasis Research Group, 2000). Arg333 (c1 65) in Region 1 of our interface model (FIG. 6) interacts with the Asp560 residue of the A2 subunit, and mutations in the Arg333 (c165) residue that eliminate the charge (e.g., Arg Glu or Leu) cause severe hemophilia B (Green et al., 2000). Further, Asn346 (c178) of factor IXa interacts with both Lys570 and Glu445 of the A2 subunit, and a mutation of Asn346 (c178) to Asp causes hemophilia B (Green et al., 2000). Similarly, Arg403 (c233) in our model interacts with Glu633 of the A2 subunit and mutations in Arg403 (c233) to Trp or Gln cause hemophilia B (Green et al., 2000). Moreover, Arg338 (c 170) of factor IXa interacts with Asp560 of the A2 subunit and mutations in both of these residues result in hemophilia (Hemostasis Research Group, 2000; Green et al., 2000). In addition, Arg562 contained within the A2 558-565 peptide region is cleaved by activated protein C (Fay et al., 1991b) and factor IXa selectively protects this site from cleavage (Regan et al., 1994). In support of this observation, Arg562 of the A2 subunit along with Gln561 interacts with Asp332 (c164) in our interface model and the mutation Asp332 (c164) to Tyr results in hemophilia B (Green et al., 2000).

Mutations in the hydrophobic patch of the interface model are also known to cause bleeding diathesis. Thus, change of Phe378 (c208) to Val or Leu in factor IXa causes hemophilia B (Green et al., 2000), and change of Ile566 to Thr in the A2 subunit causes hemophilia A (Hemostasis Research Group, 2000). Moreover, change of Phe302 (c133) to Ala has been shown to impair the interaction of factor IXa with factor VIIIa (Kolkman et al., 1999). The change of Phe302 (c133) to Ala and Phe378 (c208) to Val or Leu are expected to diminish the hydrophobic interactions involving Ile566 and Met567 of the A2 subunit. Although the change of Ile566 to Thr in the A2 subunit yields a dysfunctional factor VIII by creation of a new N-linked glycosylation site at Asn564 (Hemostasis Research Group, 2000) this mutation would also diminish hydrophobic interactions.

Concluding Remarks.

Previous studies have indicated that the helix 330 (c162) of the protease domain (Mather and Bajaj, 1999) and 558-565 region of the A2 subunit (Fay and Koshibu, 1998) represent important determinants for the interaction of IXa and VIIIa, respectively. However, it was not known whether these two regions interact with each other in the IXa/VIIIa complex. The present study provides evidence that these two regions form an interface and interact with each other through hydrophobic as well as electrostatic forces (region 1 in FIG. 6B). Modeling of the interface indicates that two other regions (regions 2 and 3) also participate in the interaction of IXa with VIIIa. Several mutations in the proposed interface cause hemophilia A or B and are known to impair the IXa:VIIIa interaction. Thus, our interface model is compatible with the existing biochemical as well as with the two-dimensional electron crystallography data of Stoylova et al. (1999). We would expect that the three-dimensional cocrystal structure of the factor IXa protease domain and the A2 subunit would support this view.

### EXAMPLE 2

### Polypeptides that are Envisioned to Function as Anti-Coagulation Agents

Determination of the EC₅₀ of for Factor IXa-Factor VIIIa Interaction in the Tenase Complex

These experiments are performed with normal factor IXa in the presence of phospholipid (PL) vesicles. 50-μl reaction mixtures in TBS-BSA (50 mM Tris-HCl, pH 7.4, 1 mg/ml BSA) with 5 mM CaCl₂ and 10 μM PL are prepared containing a fixed concentration of factor VIIIa (0.07 nM) and various concentrations of factor IXa (0-20 nM) at a constant concentration of factor X (480 nM), which is added last to initiate the reaction. The reaction is carried out at 37° C. for 30-120 seconds at which time 1 μl of 0.5 M EDTA is added to stop further generation of factor Xa. A 40 μl aliquot is added to a 0.1-mL quartz cuvette containing a synthetic substrate S-2222 in 75 μl of TBS-BSA, pH 7.4. The final concentration of S-2222 is 100 μM. The p-nitroaniline release is measured continuously (ΔA₄₀₅/min) for up to 20 minutes (Mathur et al., 1997). Factor Xa generated is calculated from a standard curve constructed using factor Xa prepared by insolubilized Russell's viper venom. In control experiments, at each factor X concentration used, the rate of factor Xa generation is also measured at various concentrations of normal factor IXa in the absence of factor VIIIa; these control values are ˜5% of the experimental values in the presence of factor VIIIa and are subtracted before analysis of the data. The EC₅₀ (functional Kd) is defined as the free concentration of normal factor IXa which provides 50% of the Vₘₐₓ. The EC₅₀ is obtained by fitting the data to a single site ligand binding equation (Eq. 1) by non-linear regression analysis using the program GraFit from Erithacus Software.

$$V = \frac{{V}_{max} L}{{EC}_{50} + L} \qquad ( Eq . 1 )$$

Where V is the rate of formation of factor Xa at a given concentration of normal factor IXa, denoted by L, and Vₘₐₓ is the rate of factor Xa formation by the factor IXa:factor VIIIa complex. The EC₅₀ obtained under above conditions is ˜0.4 nM. This EC₅₀ value is used to calculate the IC value for each competitor (e.g., peptide) employed.

Determination of the Apparent Kd_{peptide} of Binding of (1) Factor VIIIa to Each Factor IXa Peptides (SEQ ID NOS:13-16) and Factor IXa to Each of Factor VIIIa Peptides (SEQ ID NOS:9-12)

The apparent Kd (termed Kd_{peptide}) for binding of (1) factor VIIIa to the factor IXa peptides of SEQ ID NO:13, 14, 15 or 16; or of (2) factor IXa to the factor Via peptides of SEQ ID NO:9, 10, 11 or 12 is determined by its ability to inhibit the IXa:VIIIa interaction as measured by reduction in the rate of factor X activation in the tenase system. The reaction mixture for normal factor IXa contains 0.2 nM factor IXa, 0.07 nM factor VIIIa, 480 nM factor X, and 10 μM PL in TBS/BSA, pH 7.4 with 5 mM CaCl₂ and increasing amounts of the peptide. The amount of factor Xa generated is determined by hydrolysis of S-2222. The IC₅₀ (concentration of inhibitor required for 50% inhibition) is determined by fitting the data to IC₅₀ four-parameter logistic equation of Halfman (1981) given below.

$$y = \frac{a}{1 + {( x / {IC}_{50} )}^{s}} + background \qquad ( Eq . 2 )$$

y is the rate of factor Xa formation in the presence of a given concentration of inhibitory peptide represented by x, a is the maximum rate of factor Xa formation in the absence of inhibitory peptide, and s is the slope factor. Each point is weighted equally, and the data are fitted to Eq. 2 using the nonlinear regression analysis program GraFit from Erithacus Software. The background value represents ˜5% of the maximum rate of factor Xa formation in the absence of an inhibitory peptide. To obtain an apparent Kd_{peptide} value for the interaction of Factor VIIIa with Factor IXa, the following equation is employed as described by Cheng and Prusoff (1973) and further elaborated by Craig (1993)

$${Kd}_{peptide} = \frac{{IC}_{50}}{1 + ( A / {EC}_{50} )} \qquad ( Eq . 3 )$$

A is the concentration of normal factor IXa (0.2 nM), and EC₅₀ is the concentration of normal factor IXa that gives a 50% maximum response in the absence of the competitor (0.4 nM) at a specified concentration of factor X (480 nM) used in the experiment.

The inventor envisions that any one of the peptides of SEQ ID NOS:9-16 inhibits the interaction of IXa with the factor VIIIa A2 subunit at an IC₅₀ value of less than 400 μM.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

In view of the above, it will be seen that the several advantages of the invention are achieved and other advantages attained. As various changes could be made in the above methods and compositions without departing from the scope of the invention, it is intended that all matter contained in the above description and shown in the accompanying drawings shall be interpreted as illustrative and not in a limiting sense.

What is claimed is:

1. A polypeptide comprising at least 3 contiguous amino acids of a sequence that is at least 88% identical to SEQ ID NO:3 or SEQ ID NO:6, wherein said polypeptide (a) inhibits the interaction of blood coagulation factor VIIIa with blood coagulation factor IXa, (b) inhibits the activation of blood coagulation factor X, or (c) inhibits blood coagulation.

2. The polypeptide of claim 1, wherein the sequence is SEQ ID NO:3 or SEQ ID NO:6.

3. The polypeptide of claim 2, wherein the amino acid sequence is at least 5 amino acids long.

4. The agent of claim 3, wherein the amino acid sequence is at least 10 amino acids long.

5. The polypeptide of claim 4, wherein the amino acid sequence comprises a sequence selected from the group consisting SEQ ID NO:9, SEQ ED NO:10, SEQ ID NO:11, SEQ ID NO:12, SEQ ID NO:13, SEQ ID NO:14, SEQ ID NO:15, and SEQ ID NO:16.

6. The polypeptide of claim 5, wherein the amino acid sequence consists essentially of a sequence selected from the group consisting SEQ ID NO:9, SEQ ID NO:10, SEQ ID NO:11, SEQ ID NO:12, SEQ ID NO:13, SEQ ED NO:14, SEQ ID NO:15, and SEQ ID NO:16.

7. An agent comprising an antibody binding site, wherein (a) the antibody binding site specifically binds to region 2 or 3 of blood coagulation factor VIIIa or to region 2 or 3 of blood coagulation factor IXa, and (b) the agent (i) inhibits the interaction of blood coagulation factor VIIIa with blood coagulation factor IXa, (ii) inhibits the activation of blood coagulation factor x or (iii) inhibits blood coagulation.

8. The agent of claim 7, wherein the antibody binding site specifically binds to the amino acid sequence of any one of claims 1-6.

9. The agent of claim 8, wherein the agent is an antibody.

10. The agent of claim 9, wherein the agent is a monoclonal antibody.

11. The agent of claim 10, wherein the monoclonal antibody is a humanized monoclonal antibody.

12. A polynucleotide encoding an amino acid sequence of any one of the polypeptides of claims 1-6, wherein the polynucleotide is operably linked to a control sequence that allows the polynucleotide to be translated in a mammalian cell.

13. A pharmaceutical composition comprising (a) the polypeptide of any one of claims 1-6, (b) the agent of any one of claims 7-11, or (c) the polynucleotide of claim 15, in a pharmaceutically acceptable excipient.

14. The pharmaceutical composition of claim 13, wherein the excipient is suitable for intravenous administration.

15. A method of treatment to prevent coagulation in a patient in need thereof, the method comprising administering to the patient the pharmaceutical composition of claim 13 or 14.

16. The method of claim 15, wherein the patient is suffering from a cardiovascular disorder.

17. The method of claim 16, wherein the cardiovascular disorder is selected from the group consisting of thrombosis, atherosclerosis and restenosis.

18. The method of any one of claims 15-17, wherein the pharmaceutical composition is administered intravenously.

19. A method for identifying a compound having anti-coagulation activity, the method comprising determining whether a compound displaces the interaction of the polypeptide of any one of claims 1-7 to factor VIIIa or factor IXa.

20. The method of claim 19, wherein the polypeptide is labeled with a detectable marker.

21. The method of claim 20, wherein the detectable marker is selected from the group consisting of a fluorescent marker, a radioactive marker, and a spin label.

22. A method of preventing coagulation in a blood sample, comprising (a) adding the polypeptide of any of claims 1-6 to the sample, or (b) adding the agent of any of claims 7-11 to the sample.
